(19) United States
(12) Patent Application Publication
(10) Pub. No.: US 2026/0232627 A1
(43) Pub. Date: Aug. 13, 2026

(54) UROLITHIN FORMULATIONS

(71) Applicant: Amazentis SA, Lausanne (CH)

(72) Inventors: Christopher L. Rinsch, Lausanne (CH); Davide D'Amico, Renens (CH); Julie Faitg, Lausanne (CH)

(21) Appl. No.: 19/409,178

(22) Filed: Dec. 4, 2025

(30) Foreign Application Priority Data
Dec. 4, 2024 (GB) 2417829.5

(51) Int. Cl. A61K 31/37

(52) U.S. Cl. CPC A61K 31/37

(57) ABSTRACT

The invention relates to formulations of urolithins comprising urolithins and pectin and such formulations of urolithins and pectin, further comprising glycerol. The invention further comprises formulations comprising urolithins and Gum Arabic. The invention further comprises such formulations comprising further excipients and processes for the preparation of such formulations. The invention further comprises methods of using such formulations in the treatment of conditions and diseases and for the promotion of health and performance, such in the improving muscle function. The invention further comprises chewable formulations and solutions comprising such formulations.

### RELATED APPLICATION

This application claims the benefit of priority to UK Patent Application No. GB 2417829.5, filed Dec. 4, 2024.

### BACKGROUND

Urolithins have been proposed as treatments for a variety of conditions related to inadequate mitochondrial activity, including obesity, reduced metabolic rate, metabolic syndrome, diabetes mellitus, cardiovascular disease, hyperlipidaemia, neurodegenerative diseases, cognitive disorders, mood disorders, stress, and anxiety disorders; for weight management, or to increase muscle performance or mental performance. See WO2012/088519 (Amazentis SA). In WO2007/127263 (The Regents of the University of California), the use of urolithins for the treatment of various neoplastic diseases is described.

International patent publication WO2014/004902 (derived from application PCT/US2013/48310) discloses a method of increasing autophagy, including specifically mitophagy, in a cell, comprising contacting a cell with an effective amount of a urolithin or a pharmaceutically acceptable salt thereof, thereby increasing autophagy, including specifically mitophagy, in the cell. Administration may be to a subject having a disease or condition selected from metabolic stress, cardiovascular disease, endothelial cell dysfunction, sarcopenia, muscle degenerative disease, Duchenne muscular dystrophy, alcoholic liver disease, non-alcoholic fatty liver disease, drug-induced liver or muscle injury, α1-antitrypsin deficiency, ischemia/reperfusion injury, inflammation, aging of the skin, inflammatory bowel disease, Crohn's disease, obesity, metabolic syndrome, type II diabetes mellitus, hyperlipidaemia, osteoarthritis, neurodegenerative disease, Alzheimer's disease, Huntington's disease, Parkinson's disease, amyotrophic lateral sclerosis, age-related macular degeneration, mitochondrial diseases (including for example poor growth, loss of muscle coordination, muscle weakness, visual problems, hearing problems, heart disease, liver disease, kidney disease, gastrointestinal disorders, respiratory disorders, neurological problems, autonomic dysfunction sometimes learning disabilities, and dementia (as a result of mitochondrial disease), muscle diseases; cancer, cognitive disorder, stress, and mood disorder.

Chewable products (gummy formulations), generally made of a gelatine or a pectin matrix with sugar, glucose, corn syrup, flavouring, colouring and citric acid have been a popular snack food product. The product (formulation) typically has a gel or gel-like structure and texture, and is produced in a variety of shapes, colours and flavours that are chewable when consumed. Recently, gummy products have been supplemented with vitamins, minerals, essential oils and other nutritional supplements to provide a nutritional supplement that appeals to children and adults that do not like to swallow or have difficulty swallowing tablets or capsules.

Gelatine is a soluble and gelatinous protein that is extracted from animal bones, skin, tendons and muscles, and then partially hydrolyzed by acid, alkali or enzymes. Gelatine has been extensively used in chewable formulations, however, more recently there has been a decrease in use of gelatine. This has been advocated due to its animal origin, since traditionally, vegetarian consumers and their religious advocate of avoiding animal-derived products. More recently, although ordinary consumers do not concern vegetarianism or religious preferences, they also tend to prefer foods in which gelatine is replaced with another agent, for instance gums extracted from plants, for example, pectin.

However, although plant-based chewable formulations, such as formulations, comprising pectin, are a popular option they can be difficult to manufacture. Therefore, there is an on-going need for improvements in chewable formulations comprising pectin, especially to formulate novel active ingredients as chewable formulations.

### SUMMARY

The invention relates to formulations of urolithins comprising urolithins and pectin and such formulations of urolithins and pectin, further comprising glycerol. The invention further comprises formulations comprising urolithins and Gum Arabic. The invention further comprises such formulations comprising further excipients and processes for the preparation of such formulations. The invention further comprises methods of using such formulations in the treatment of conditions and diseases and for the promotion of health and performance, such in the improving muscle function. The invention further comprises chewable formulations and solutions comprising said formulations.

According to a one aspect of the invention there is provided a formulation comprising:
a) a compound of formula (I) or a salt thereof:

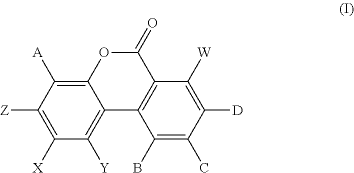

wherein:
A, B, C and D are each independently selected from H and OH;
W, X and Y are each independently selected from H and OH; and
Z is selected from H and OH; and
b) Gum Arabic.
wherein the ratio of the compound of formula (I) to Gum Arabic is between about 1 to 4 (w/w) and about 15 to 1 (w/w).

### BRIEF DESCRIPTION OF THE FIGURES

FIG. 1: Dissolution profiles of powders containing 10 mg UA complexed with Citrus pectin in FaSSIF medium compared to 10 mg of pure UA powder, using EtOH as solvent. Left graph: comparison of a formulation UA/Pectin in which excipients are in a w/w ratio of 1/1. Right graph: comparison of a formulation UA/Pectin in which excipients are in a w/w ratio of 4/1.

FIG. 2: Dissolution profiles of powders containing the equivalent of 10 mg UA complexed with Citrus pectin in FaSSIF medium compared to 10 mg of pure UA powder, using water as solvent. Top left graph: comparison of a formulation UA/Pectin in which excipients are in a w/w ratio of 1/1. Top right graph: comparison of a formulation UA/Pectin in which excipients are in a w/w ratio of 4/1. Bottom left graph: comparison of a formulation UA/Pectin in which excipients are in a w/w ratio of 7/1. Bottom right graph: comparison of a formulation UA/Pectin in which excipients are in a w/w ratio of 10/1.

FIG. 3: AUC of powders containing the equivalent of 10 mg UA complexed with Citrus pectin in FaSSIF medium compared to 10 mg of pure UA powder, using water as solvent. Data expressed as mean+/−SD. Adjusted P Value ****<0.0001, One-way ANOVA.

FIG. 4: Comparison of the solubility of the different Urolithin A formulations—maximum concentration of urolithin A in solution achieved comparing formulations with UA prepared in Ethanol or in water (Cmax).

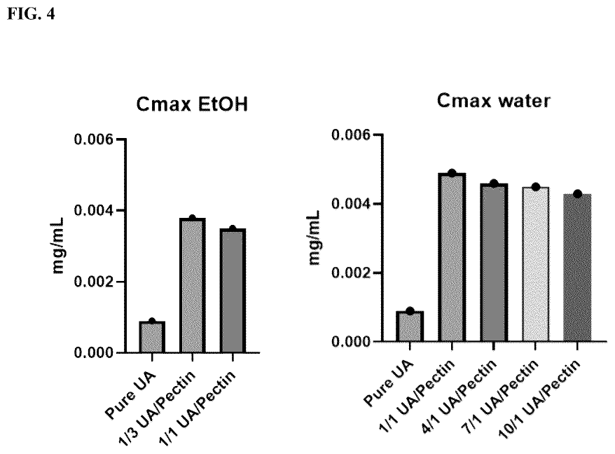

FIG. 5: Dissolution profiles of powders containing the equivalent of 10 mg UA after physical mixture (without spray drying) with Citrus pectin in FaSSIF medium compared to 10 mg of pure UA powder, using water as solvent. Top left graph: comparison of a formulation UA/Pectin in which excipients are in a w/w ratio of 1/1. Top right graph: comparison of a formulation UA/Pectin in which excipients are in a w/w ratio of 4/1. Bottom left graph: comparison of a formulation UA/Pectin in which excipients are in a w/w ratio of 7/1. Bottom right graph: comparison of a formulation UA/Pectin in which excipients are in a w/w ratio of 10/1.

FIG. 6: AUC of powders containing the equivalent of 10 mg UA added as physical mixture, without spray drying, with Citrus pectin in FaSSIF medium compared to 10 mg of pure UA powder, using water as solvent. Data expressed as mean+/−SD. Adjusted P Value ****<0.0001, One-way ANOVA.

FIG. 7: A synoptic overview of the PROCEPT spray dryer with BF nozzle.

FIG. 8: An XRPD spectrum of crystalline urolithin A

FIG. 9: An XRPD spectrum of urolithin A formulations, prepared by spray drying using water as solvent and using excipient pectin, complexed in a ratio of 1 to 1, urolithin A to excipient.

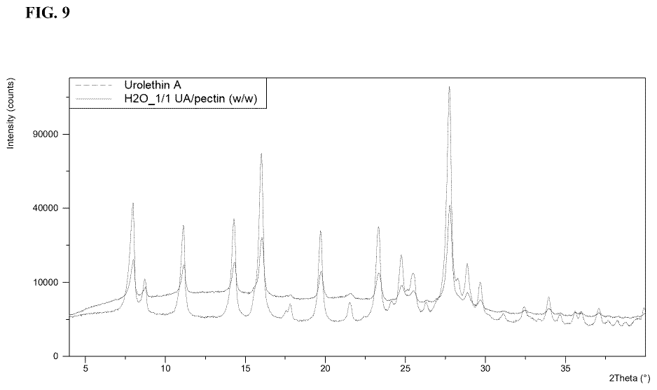

FIG. 10: An XRPD spectrum of urolithin A formulations, prepared by spray drying using water as solvent and using excipient pectin, complexed in a ratio of 4 to 1, urolithin A to excipient.

FIG. 11: Solubility of urolithin A in FaSSIF medium comparing a spray-dried and non-spray dried formulation.

FIG. 12: Solubility of urolithin A in water comparing a spray-dried comprising guar gum and non-spray dried formulation.

FIG. 13: Solubility of urolithin A in water comparing a spray-dried comprising guar gum and non-spray dried formulation.

FIG. 14: Urolithin A release comparing physical mixture with Arabic Gum and spray-dried mixtures with Arabic Gum.

FIG. 15: Comparison of control (DMSO) with pectin alone, urolithin A alone and spray dried formulations combining urolithin A and pectin on mitochondrial respiration in muscle cells.

FIG. 16A: The effect of glycerin level (VegGlycerin x-axis) on properties of Cfirmness (force required for compression, g-force), Ctoughness (work required for compression, g·force·mm).

FIG. 16B: The effect of glycerin level (VegGlycerin x-axis) on properties of Pfirmness (initial puncture force, g-force) and Ptoughness (work to continue puncture, g-force-mm).

FIG. 16C: The effect of glycerin level on the desirability to consumers.

### DETAILED DESCRIPTION

According to a one aspect of the invention there is provided a formulation comprising:
a) a compound of formula (I) or a salt thereof:

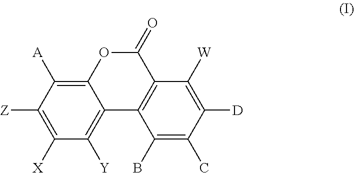

wherein:
A, B, C and D are each independently selected from H and OH;
W, X and Y are each independently selected from H and OH; and
Z is selected from H and OH;
b) pectin; and
c) glycerol.

According to a further aspect of the invention there is provided a formulation, comprising pectin, as described herein, wherein the formulation comprises a ratio of a compound of formula (I) to pectin to glycerol in the range between:
(i) about 1 to about 1 to 2 and about 10 to about 1 to 2;
(ii) about 3 to about 1 to 2 (w/w) and about 5 to about 1 to 2 (w/w);
(iii) about 4 to about 1 to 2 (w/w).

In a further aspect of the invention, there is provided a formulation comprising a compound of formula (I), pectin and glycerol, wherein the formulation comprises a ratio of a compound of formula (I) to pectin to glycerol in the range between
(i) about 1 to about 1 to 2 and about 10 to about 1 to 2;
(ii) about 2 to about 1 to 2 and about 8 to about 1 to 2;
(iii) about 3 to about 1 to 2 (w/w) and about 7 to about 1 to 2 (w/w);
(iv) about 4 to about 1 to 2 (w/w) and about 6 to about 1 to 2 (w/w);
(v) about 4 to about 1 to 2 (w/w) and about 5 to about 1 to 2 (w/w);
(vi) about 4 to about 1 to 2 (w/w).
(vii) about 1 to about 1 to 3 and about 10 to about 1 to 3;
(viii) about 2 to about 1 to 3 and about 8 to about 1 to 3;
(ix) about 3 to about 1 to 3 (w/w) and about 7 to about 1 to 3 (w/w);
(x) about 4 to about 1 to 3 (w/w) and about 6 to about 1 to 3 (w/w);
(xi) about 4 to about 1 to 3 (w/w) and about 5 to about 1 to 3 (w/w);
(xii) about 4 to about 1 to 3 (w/w);
(xiii) about 1 to about 1 to 1 and about 10 to about 1 to 1;
(xiv) about 2 to about 1 to 1 and about 8 to about 1 to 1;
(xv) about 3 to about 1 to 1 (w/w) and about 7 to about 1 to 1 (w/w);
(xvi) about 4 to about 1 to 1 (w/w) and about 6 to about 1 to 1 (w/w);
(xvii) about 4 to about 1 to 1 (w/w) and about 5 to about 1 to 1 (w/w); or
(xviii) about 4 to about 1 to 1 (w/w).

According to a further aspect of the invention there is provided a formulation, for example, a chewable formulation, comprising:
a) about 1% to about 20% (w/w) a compound of formula (I) or a salt thereof, as defined herein;
b) about 0.1% to about 10% (w/w) pectin; and
c) about 2% to about 8% (w/w) glycerol, for example about 5%.

According to a further aspect of the invention there is provided a formulation, comprising pectin, as described herein, wherein the pectin is selected from low methoxy pectin, high methoxy pectin or amidated low methoxy-pectin, or a mixture thereof, for example, wherein the pectin is low methoxypectin, such as, amidated low methoxypectin.

According to a further aspect of the invention there is provided a formulation, comprising pectin, as described herein, wherein the formulation comprises pectin in the range from about 0.1% to about 10% (w/w), for example about 3% to about 7% (w/w).

According to a further aspect of the invention, there is provided a formulation comprising:
a) a compound of formula (I) or a salt thereof:

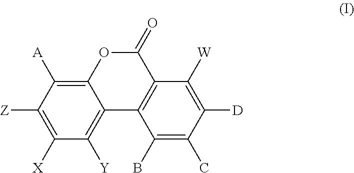

wherein:
A, B, C and D are each independently selected from H and OH;
W, X and Y are each independently selected from H and OH; and
Z is selected from H and OH; and
b) Gum Arabic;
wherein the ratio of the compound of formula (I) to Gum Arabic is between about 1 to 10 and about 15 to 1.

According to a further aspect of the invention, there is provided a formulation consisting of:
a) a compound of formula (I) or a salt thereof, as defined herein:
b) Gum Arabic;
wherein the ratio of the compound of formula (I) to Gum Arabic is between about 1 to 10 and about 15 to 1.

According to a further aspect of the invention, there is provided a formulation comprising:
a) About 9% to about 94% (w/w) of a compound of formula (I) or a salt thereof:

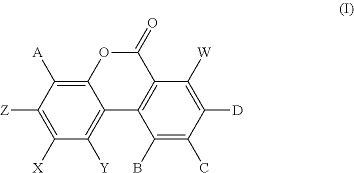

wherein:
A, B, C and D are each independently selected from H and OH;
W, X and Y are each independently selected from H and OH; and
Z is selected from H and OH; and
b) about 6% to about 91% (w/w) Gum Arabic.

According to a further aspect of the invention, there is provided a formulation consisting of:
a) about 9% to about 94% (w/w) of a compound of formula (I) or a salt thereof, as defined herein:
b) about 6% to about 91% (w/w) Gum Arabic.

According to a further aspect of the invention, there is provided a formulation comprising Gum Arabic, as described therein, further comprising:
c) glycerol.

According to a further aspect of the invention there is provided a formulation, comprising Gum Arabic, as described herein, wherein the Gum Arabic is selected from the exudate, for example, dried exudate, from the trunks and/or branches of Acacia Senegal, Vachellia (Acacia) seyal, Acacia polyacantha, Acacia laeta and/or Acacia mellifera, for example, the exudate is from the trunks and/or branches of Acacia Senegal and/or Vachellia (Acacia) seyal.

According to a further aspect of the invention, there're is provided a formulation comprising or consisting of Gum Arabic as described herein wherein the ratio of the compound of formula (I) to Gum Arabic is between:
(i) about 1 to 10 and about 15 to 1 (w/w);
(ii) about 1 to 8 to about 15 to 1 (w/w)
(iii) about 1 to 6 to about 15 to 1 (w/w)
(iv) about 1 to 4 and about 15 to 1 (w/w);
(v) about 1 to 2 to about 15 to 1 (w/w)
(vi) about 1 to 1 to about 15 to 1 (w/w)
(vii) about 1 to 10 and about 10 to 1 (w/w);
(viii) about 1 to 8 to about 15 to 1 (w/w)
(ix) about 1 to 6 to about 10 to 1 (w/w)
(x) about 1 to 4 and about 10 to 1 (w/w);
(xi) about 1 to 3 to about 10 to 1 (w/w);
(xii) about 1 to 2 to about 10:1 (w/w)
(xiii) about 1 to 1 to about 10:1 (w/w)
(xiv) about 1 to 4 to about 8 to 1 (w/w);
(xv) about 1 to 4 to about 5 to 1 (w/w);
(xvi) about 1 to 4 to about 4 to 1 (w/w);
(xvii) about 1 to 4 to about 3 to 1 (w/w);
(xviii) about 1 to 2 to about 4 to 1 (w/w)
(xix) about 1 to 1 to about 4 to 1 (w/w)
(xx) about 1 to 4 to about 6 to 1 (w/w);
(xxi) about 1 to 3 to about 6 to 1 (w/w)
(xxii) about 1 to 2 to about 6 to 1 (w/w)
(xxiii) about 1 to 1 to about 6 to 1 (w/w)
(xxiv) about 1 to 10 to about 5 to 1 (w/w)
(xxv) about 1 to 6 to about 5 to 1 (w/w)
(xxvi) about 1 to 4 to about 5 to 1 (w/w);
(xxvii) about 1 to 3 to about 5 to 1 (w/w)
(xxviii) about 1 to 2 to about 5 to 1 (w/w)
(xxix) about 1 to 1 to about 5 to 1 (w/w);
(xxx) about 2 to 1 to about 5 to 1 (w/w);
(xxxi) about 3 to 1 to about 5 to 1 (w/w);
(xxxii) about 4 to 1 (w/w);
(xxxiii) about 1 to 2 to about 10 to 1 (w/w).

According to a further aspect of the invention, there're is provided a formulation comprising or consisting of Gum Arabic as described herein wherein the ratio of the compound of formula (I) to Gum Arabic is between:
(i) about 1 to 5 and about 10 to 1 (w/w);
(ii) about 1 to 4 to about 10 to 1 (w/w);
(iii) about 1 to 2 to about 10 to 1 (w/w)
(iv) about 1 to 1 to about 10:1 (w/w);
(v) about 2 to 1 to about 8:1 (w/w);
(vi) about 2 to 1 to about 6:1 (w/w); or
(vii) about 3 to 1 to about 5 to 1 (w/w).

According to a further aspect of the invention there is provided a formulation, comprising Gum Arabic, as described herein, wherein the formulation comprises the compound of formula (I) in the range from:
(i) about 9% to about 94% (w/w);
(ii) about 20% to about 90% (w/w)
(iii) about 30% to about 90% (w/w);
(iv) about 40% to about 90% (w/w);
(v) about 50% to about 90% (w/w);
(vi) about 60% to about 90% (w/w);
(vii) about 70% to about 90% (w/w);
(viii) about 75% to about 85% (w/w); or
(ix) about 80% (w/w).

According to a further aspect of the invention there is provided a formulation, comprising Gum Arabic, as described herein, wherein the formulation comprises Gum Arabic in the range from:
(i) about 6% to about 91% (w/w),
(ii) about 10% to about 80% (w/w);
(iii) about 10% to about 70% (w/w)
(iv) about 10% to about 60% (w/w)
(v) about 10% to about 50% (w/w);
(vi) about 10% to about 40% (w/w);
(vii) about 10% to about 30% (w/w)
(viii) about 25% (w/w)
(ix) about 20% (w/w)
(x) about 15% (w/w)

According to a further aspect of the invention, there is provided a formulation comprising Gum Arabic, as described therein, wherein the formulation comprises Gum Arabic in the range in the range about 1% to about 10% (w/w), for example, about 2% to about 8% (w/w), for example, about 4% to about 8% (w/w), for example, about 4% (w/w), about 5% (w/w), about 6% (w/w), about 7% (w/w) or about 8% (w/w).

According to a further aspect of the invention, there is provided a formulation comprising Gum Arabic, as described therein, wherein the formulation comprises Urolithin A and Gum Arabic in a weight ratio of about 4:1 to 15:1 (w/w), for example, about 1:1 to 9:1 (w/w).

According to a further aspect of the invention, there is provided a chewable formulation comprising Gum Arabic, as described therein, comprising about 1% to about 6% (w/w) of the final formulation.

According to a further aspect of the invention, there is provided an aqueous formulation comprising Gum Arabic, as described therein, comprising Urolithin A and Gum Arabic in a weight ratio of about 4:1 to about 15:1, for example, about 1:1 to about 9:1.

Gum Arabic comprising formulations of the invention can be used to make several types of formulations, for example, powder formulations, topical formulations, gummy formulations and aqueous formulations. Urolithins, such as urolithin A, are hard to formulate due to the low solubility of urolithins. However, one particularly surprising aspect of the invention is the ability to make aqueous formulations using Gum Arabic and urolithins, such as urolithin A, such as spray-dried formulations comprising Gum Arabic and urolithins, such as urolithin A.

Therefore, according to a further aspect of the invention, there is provided a spray-dried formulation, comprising:
(a) a compound of formula (I), as defined herein, for example, urolithin A; and (b) Gum Arabic.

According to a further aspect of the invention, there is provided an aqueous formulation, comprising a formulation, for example, a spray-dried formulation comprising
(a) a compound of formula (I), as defined herein, for example, urolithin A; and
(b) Gum Arabic;
admixed with water.

According to a further aspect of the invention, there is provided an aqueous formulation, comprising a formulation, for example, a spray-dried formulation comprising
(a) a compound of formula (I), as defined herein, for example, urolithin A; and
(b) Gum Arabic;
admixed with water, wherein the compound of formula (I) to Gum Arabic is in the range: about 1 to 4 (w/w) to about 15 to 1 (w/w) for example about 4 to 1 (w/w).

In a further embodiment of the invention, there is provided a Gum Arabic formulation of the invention, wherein the formulation is a powder formulation.

In a further embodiment of the invention, there is provided a Gum Arabic formulation of the invention, wherein the formulation is a topical formulation.

According to a one aspect of the invention there is provided a chewable formulation comprising:
a) a gelling component, and
b) a compound of formula (I) or a salt thereof:

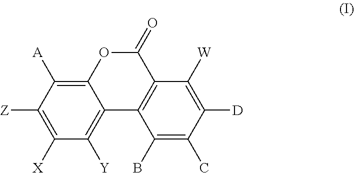

wherein:
A, B, C and D are each independently selected from H and OH;
W, X and Y are each independently selected from H and OH; and
Z is selected from H and OH.

According to a further aspect of the invention, there is provided a chewable formulation, comprising:
a) a gelling component,
b) a compound of formula (I) or a salt thereof:
wherein the formulation does not include a sweetener system.

According to a further aspect of the invention, there is provided a chewable formulation, comprising:
a) a gelling component,
b) a compound of formula (I) or a salt thereof:
c) a sweetener system, wherein the sweetener system comprises one or more artificial sweeteners.

According to a further aspect of the invention, there is provided a chewable formulation, comprising:
a) a gelling component,
b) a compound of formula (I) or a salt thereof:
c) a sweetener system, wherein the sweetener system comprises allulose.

According to a further aspect of the invention, there is provided a chewable formulation, comprising:
a) a gelling component,
b) a compound of formula (I) or a salt thereof:
c) a sweetener system, wherein the sweetener system comprises inulin.

According to a further aspect of the invention, there is provided a chewable formulation, comprising:
a) a gelling component,
b) a compound of formula (I) or a salt thereof:
c) a sweetener system, wherein the sweetener system comprises trehalose.

According to a further aspect of the invention, there is provided a chewable formulation, comprising:
a) a gelling component,
b) a compound of formula (I) or a salt thereof:
c) a sweetener system, wherein the sweetener system consists of one or more artificial sweeteners.

According to a further aspect of the invention, there is provided a chewable formulation, comprising:
a) a gelling component,
b) a compound of formula (I) or a salt thereof, and
c) a pH buffer.

According to a further aspect of the invention, there is provided a chewable formulation, comprising:
a) a gelling component,
b) a compound of formula (I) or a salt thereof, and
c) a pH between about 2.7 and about 3.7.

According to a further aspect of the invention, there is provided a chewable formulation, comprising:
a) a gelling component,
b) a compound of formula (I) or a salt thereof, and
c) a pH buffer, wherein the pH buffer maintains the pH between about 2.7 and about 3.7.

According to a further aspect of the invention, there is provided a chewable formulation, comprising:
a) a gelling component,
b) a compound of formula (I) or a salt thereof, and
c) a pH between about 2.7 and about 4.0.

According to a further aspect of the invention, there is provided a chewable formulation, comprising:
a) a gelling component,
b) a compound of formula (I) or a salt thereof, and
c) a pH buffer, wherein the pH buffer maintains the pH between about 2.7 and about 4.0.

According to a further aspect of the invention, there is provided a chewable formulation, comprising:
a) a gelling component,
b) a compound of formula (I) or a salt thereof, and
c) a fibre component, such as a soluble fibre component.

According to a further aspect of the invention, there is provided a chewable formulation, comprising:
a) a gelling component,
b) a compound of formula (I) or a salt thereof, and
c) a brix between about 75 degrees to about 85 degrees for example, about 77 degrees to about 82 degrees.

According to a further aspect of the invention, there is provided a chewable formulation, comprising:
a) a gelling component,
b) a compound of formula (I) or a salt thereof, and
c) a terminal boiling point between about 108° C. and about 111° C. (about 228° F. to about 231° F.).

According to a further aspect of the invention, there is provided a low-calorie chewable formulation, comprising:
a) a gelling component,
b) a compound of formula (I) or a salt thereof, and
wherein the formulation comprises less than about 30% (w/w) sugar.

According to a further aspect of the invention, there is provided a low-calorie chewable formulation, comprising:
a) a gelling component,
b) a compound of formula (I) or a salt thereof, and
wherein the formulation comprises less than about 50% (w/w) sugar, for example less than about 45% (w/w) sugar, such as less than about 40% (w/w) or less than about 30% (w/w) sugar.

According to a further aspect of the invention, there is provided a low-calorie chewable formulation, comprising:
a) a gelling component,
b) a compound of formula (I) or a salt thereof, and
wherein the formulation comprises about 25% to about 40% sugar.

According to a further aspect of the invention, there is provided a chewable formulation, comprising:
a) a gelling component,
b) a compound of formula (I) or a salt thereof, and
c) wherein the gelling component comprises pectin with an esterification range of about 55% to about 75%, for example, 60% to about 68%.

According to a further aspect of the invention, there is provided a chewable formulation, comprising:
a) a gelling component, comprising a high-methoxy pectin, and
b) a compound of formula (I) or a salt thereof.

According to a further aspect of the invention, there is provided a chewable formulation, comprising:
a) a gelling component, comprising a low-methoxy pectin, and
b) a compound of formula (I) or a salt thereof.

According to a further aspect of the invention, there is provided a chewable formulation, comprising:
a) a gelling component, comprising an amidated low-methoxy pectin, and
b) a compound of formula (I) or a salt thereof.

According to a further aspect of the invention, there is provided a chewable formulation, comprising:
a) a gelling component, comprising a low-methoxy pectin,
b) a compound of formula (I) or a salt thereof, and
c) a divalent cation, for example, provided by a calcium salt, such a calcium salt selected from calcium chloride, calcium citrate and/or calcium sulphate.

According to a further aspect of the invention, there is provided a formulation, comprising:
a) pectin,
b) a compound of formula (I) or a salt thereof, as defined herein;
c) glycerol;
wherein the formulation does not include a sweetener system.

According to a further aspect of the invention, there is provided a formulation, comprising:
a) pectin,
b) a compound of formula (I) or a salt thereof:
c) glycerol
d) a sweetener system, wherein the sweetener system comprises one or more artificial sweeteners.

According to a further aspect of the invention, there is provided a formulation, comprising:
a) pectin;
b) a compound of formula (I) or a salt thereof:
c) glycerol;
d) a sweetener system, wherein the sweetener system comprises allulose.

According to a further aspect of the invention, there is provided a formulation, comprising:
a) pectin,
b) a compound of formula (I) or a salt thereof:
c) glycerol;
d) a sweetener system, wherein the sweetener system comprises inulin.

According to a further aspect of the invention, there is provided a chewable formulation, comprising:
a) pectin,
b) a compound of formula (I) or a salt thereof:
c) glycerol;
d) a sweetener system, wherein the sweetener system comprises trehalose.

According to a further aspect of the invention, there is provided a cheable formulation, comprising:
a) pectin,
b) a compound of formula (I) or a salt thereof:
c) glycerol;
d) a sweetener system, wherein the sweetener system consists of one or more artificial sweeteners.

According to a further aspect of the invention, there is provided a formulation, comprising:
a) pectin,
b) a compound of formula (I) or a salt thereof,
c) glycerol; and
d) a pH buffer.

According to a further aspect of the invention, there is provided a formulation, comprising:
a) pectin,
b) a compound of formula (I) or a salt thereof,
c) glycerol; and
d) a pH between about 2.7 and about 3.7.

According to a further aspect of the invention, there is provided a formulation, comprising:
a) pectin,
b) a compound of formula (I) or a salt thereof,
c) glycerol; and
d) a pH buffer, wherein the pH buffer maintains the pH between about 2.7 and about 3.7.

According to a further aspect of the invention, there is provided a formulation, comprising:
a) pectin,
b) a compound of formula (I) or a salt thereof,
c) glycerol; and
d) a pH between about 2.7 and about 4.0.

According to a further aspect of the invention, there is provided a formulation, comprising:
a) pectin,
b) a compound of formula (I) or a salt thereof,
c) glycerol; and
d) a pH buffer, wherein the pH buffer maintains the pH between about 2.7 and about 4.0.

According to a further aspect of the invention, there is provided a formulation, comprising:
a) pectin,
b) a compound of formula (I) or a salt thereof,
c) glycerol; and
d) a fibre component, such as a soluble fibre component.

According to a further aspect of the invention, there is provided a formulation, comprising:
a) pectin,
b) a compound of formula (I) or a salt thereof,
c) glycerol; and
d) a brix between about 75 degrees to about 85 degrees for example, about 77 degrees to about 82 degrees.

According to a further aspect of the invention, there is provided a formulation, comprising:
a) pectin,
b) a compound of formula (I) or a salt thereof,
c) glycerol; and
d) a terminal boiling point between about 108° C. and about 111° C. (about 228° F. to about 231° F.).

According to a further aspect of the invention, there is provided a low-calorie formulation, comprising:
a) pectin,
b) a compound of formula (I) or a salt thereof,
c) glycerol; and
wherein the formulation comprises less than about 30% (w/w) sugar.

According to a further aspect of the invention, there is provided a low-calorie formulation, comprising:
a) pectin,
b) a compound of formula (I) or a salt thereof,
c) glycerol; and
wherein the formulation comprises less than about 50% (w/w) sugar, for example less than about 45% (w/w) sugar, such as less than about 40% (w/w) or less than about 30% (w/w) sugar.

According to a further aspect of the invention, there is provided a low-calorie formulation, comprising:
a) pectin,
b) a compound of formula (I) or a salt thereof,
c) glycerol; and
wherein the formulation comprises about 25% to about 40% sugar.

According to a further aspect of the invention, there is provided a formulation, comprising:
a) pectin,
b) a compound of formula (I) or a salt thereof,
c) glycerol; and
d) wherein the gelling component comprises pectin with an esterification range of about 55% to about 75%, for example, 60% to about 68%.

According to a further aspect of the invention, there is provided a formulation, comprising:
a) pectin, comprising a low-methoxy pectin,
b) a compound of formula (I) or a salt thereof,
c) glycerol; and
d) a divalent cation, for example, provided by a calcium salt, such a calcium salt selected from calcium chloride, calcium citrate and/or calcium sulphate.

According to a further aspect of the invention, there is provided a formulation, comprising:
a) Gum Arabic,
b) a compound of formula (I) or a salt thereof, as defined herein;
c) optionally glycerol;
wherein the formulation does not include a sweetener system.

According to a further aspect of the invention, there is provided a formulation, comprising:
a) Gum Arabic,
b) a compound of formula (I) or a salt thereof:
c) optionally glycerol
d) a sweetener system, wherein the sweetener system comprises one or more artificial sweeteners.

According to a further aspect of the invention, there is provided a formulation, comprising:
a) Gum Arabic;
b) a compound of formula (I) or a salt thereof:
c) optionally glycerol;
d) a sweetener system, wherein the sweetener system comprises allulose.

According to a further aspect of the invention, there is provided a formulation, comprising:
a) Gum Arabic,
b) a compound of formula (I) or a salt thereof:
c) optionally glycerol;
d) a sweetener system, wherein the sweetener system comprises inulin.

According to a further aspect of the invention, there is provided a chewable formulation, comprising:
a) Gum Arabic,
b) a compound of formula (I) or a salt thereof:
c) optionally glycerol;
d) a sweetener system, wherein the sweetener system comprises trehalose.

According to a further aspect of the invention, there is provided a cheable formulation, comprising:
a) Gum Arabic,
b) a compound of formula (I) or a salt thereof:
c) optionally glycerol;
d) a sweetener system, wherein the sweetener system consists of one or more artificial sweeteners.

According to a further aspect of the invention, there is provided a formulation, comprising:
a) Gum Arabic,
b) a compound of formula (I) or a salt thereof,
c) optionally glycerol; and
d) a pH buffer.

According to a further aspect of the invention, there is provided a formulation, comprising:
a) Gum Arabic,
b) a compound of formula (I) or a salt thereof,
c) optionally glycerol; and
d) a pH between about 2.7 and about 3.7.

According to a further aspect of the invention, there is provided a formulation, comprising:
a) Gum Arabic,
b) a compound of formula (I) or a salt thereof,
c) optionally glycerol; and
d) a pH buffer, wherein the pH buffer maintains the pH between about 2.7 and about 3.7.

According to a further aspect of the invention, there is provided a formulation, comprising:
a) Gum Arabic,
b) a compound of formula (I) or a salt thereof,
c) optionally glycerol; and
d) a pH between about 2.7 and about 4.0.

According to a further aspect of the invention, there is provided a formulation, comprising:
a) Gum Arabic,
b) a compound of formula (I) or a salt thereof,
c) optionally glycerol; and
d) a pH buffer, wherein the pH buffer maintains the pH between about 2.7 and about 4.0.

According to a further aspect of the invention, there is provided a formulation, comprising:
a) Gum Arabic,
b) a compound of formula (I) or a salt thereof,
c) optionally glycerol; and
d) a fibre component, such as a soluble fibre component.

According to a further aspect of the invention, there is provided a formulation, comprising:
a) Gum Arabic,
b) a compound of formula (I) or a salt thereof,
c) optionally glycerol; and
d) a brix between about 75 degrees to about 85 degrees for example, about 77 degrees to about 82 degrees.

According to a further aspect of the invention, there is provided a formulation, comprising:
a) Gum Arabic,
b) a compound of formula (I) or a salt thereof,
c) optionally glycerol; and
d) a terminal boiling point between about 108° C. and about 111° C. (about 228° F. to about 231° F.).

According to a further aspect of the invention, there is provided a low-calorie formulation, comprising:
a) Gum Arabic,
b) a compound of formula (I) or a salt thereof,
c) optionally glycerol; and
wherein the formulation comprises less than about 30% (w/w) sugar.

According to a further aspect of the invention, there is provided a low-calorie formulation, comprising:
a) Gum Arabic,
b) a compound of formula (I) or a salt thereof,
c) optionally glycerol; and
wherein the formulation comprises less than about 50% (w/w) sugar, for example less than about 45% (w/w) sugar, such as less than about 40% (w/w) or less than about 30% (w/w) sugar.

According to a further aspect of the invention, there is provided a low-calorie formulation, comprising:
a) Gum Arabic,
b) a compound of formula (I) or a salt thereof,
c) optionally glycerol; and
wherein the formulation comprises about 25% to about 40% sugar.

According to a further aspect of the invention, there is provided a formulation, comprising:
a) Gum Arabic, comprising a low-methoxy pectin,
b) a compound of formula (I) or a salt thereof,
c) optionally glycerol; and
d) a divalent cation, for example, provided by a calcium salt, such a calcium salt selected from calcium chloride, calcium citrate and/or calcium sulphate.

In one embodiment, the formulation comprises a calcium salt in the range of about 0.05% to about 0.1% (w/w).

A suitable gelling component is one which provides a cohesive gelled product, when used alone or used with other gelling components. A gelling component may be selected from one of more gelling agents. For example a gelling agent selected from pectin, gelatine, and modified starch. In one embodiment the gelling agent is selected from: pectin and gelatine. In a further embodiment the gelling agent comprises pectin and gelatine. In a further embodiment the gelling agent comprises pectin. In a further embodiment, the gelling component is pectin. In a further embodiment, the gelling agent comprises gellan and carrageenan.

In a further embodiment, the formulation comprises about 0.5% to about 5% (w/w) gelling component.

In a further embodiment, the formulation comprises about 0.5% to about 4% (w/w) pectin, such as about 0.5% to about 5% (w/w), about 0.5% to about 4%, about 1.5% to about 3% (w/w) pectin, such as about 1% to about 2% (w/w), such as about 1.2% to about 1.8% (w/w), such as about 1.4% (w/w), such as about 1.5% (w/w), such as about 1.6% (w/w), such as about 1.7% (w/w).

In one embodiment, the formulation is essentially free of animal-derived products, for example, essentially free of gelatine.

In a further embodiment, the gelling component is selected from one or more of: carrageenan, alginic acid, sodium alginate, xanthan gum, gellan gum, gum Arabic, guar, and locust bean gum.

In a further embodiment, the gelling agent is selected from one or more of: pectin and gelatine.

In one embodiment, the formulation comprises a gelling component comprising pectin. In a further embodiment, the formulation comprises a gelling component consisting of pectin.

A pectin suitable for formulations of the invention is any pectin which provides the necessary gel strength for formulations of the invention. In one embodiment of the invention, the pectin is a high methoxy pectin. A high methoxy pectin is one wherein at least 50% of the galacturonic acid units are esterified with a methoxy group.

In one embodiment the methoxy pectin is esterified in a range of about 50% to about 75%, for example, about 55% to about 75%, about 55% to about 70%, or about 65% to about 75% methoxypectin. In a further embodiment the esterification range is about 60% to about 68%. In a further embodiment the esterification range is about 63% to about 65%. In a further embodiment, the methoxy pectin is about 55% esterified, about 60% esterified, about 65% esterified or about 70% esterified.

In a further embodiment of the invention, the pectin is a low methoxy pectin. A low methoxy pectin is one wherein less than 50% of the galacturonic acid units are esterified with a methoxy group.

Formulations of the invention may further comprise fibre. In one embodiment, a naturally derived fibre is used, for example, one or more selected from naturally derived inulin, inulin extract, synthetic inulin, hydrolysis products of inulin commonly known as fructooligosaccharides, galacto-oligosaccharides, xylooligosaccharides, oligo derivatives of starch, husks, brans, psyllium, polysaccharides, polycarbophil, lignin, arabinogalactans, chitosans, oat fibre, soluble corn fibre, non-digestible corn dextrin, non-digestible wheat dextrin, locust bean gum and derivatives of locust bean gum, hydroxypropylmethyl cellulose (HPMC), pectin, and mixtures thereof.

In some embodiments, fibre may include inulin, wheat dextrin, or fructooligosaccharides. Inulin, wheat dextrin, and fructooligosaccharides may also act as a thickening agent and improve the texture of the formulation. Various load rates of dietary fibre can be incorporated in the formulation to create improved texture and at certain load rates can provide dietary benefits including promoting a healthy digestion system, controlling blood sugar levels, and providing probiotic benefits. The addition of the dietary fibre along with the remaining components allow for the addition of water that helps displace sugar within the flavoured chewy or gummy confection.

In a further embodiment the fibre is selection from soluble tapioca, psyllium husk powder, apple fibre, dextrins, inulin, or mixtures thereof.

In one embodiment, the fibre may present in an amount of from about 10% to about 60% (w/w), alternatively about 20% to about 60% (w/w), alternatively about 30% to about 60% (w/w), alternatively about 30% to about 50% (w/w), alternatively about 30% to about 40%, such as about 35% (w/w), such as about 37% (w/w), such as about 40% (w/w).

Conventional chewable formulations comprise no more than 6% (w/w) of active ingredients. However, through careful selection of active ingredients, formulations of the invention can comprise more than about 20% (w/w) active ingredients. In some embodiments, the formulation may include the active component from about 5% to about 25%, (w/w) active ingredients, for example, about 6% to about 25% (w/w) active ingredients, for example, about 10% to about 25% (w/w) active ingredients, for example, about 15% to about 25% (w/w) active ingredients, for example, about 10% to about 20% (w/w) or about 6% to about 20%, active ingredients, for example, about 20% (w/w) active ingredients.

In one embodiment, the chewable formulations of the invention have reduced sugar content when compared to conventional commercially available chewable formulations. For example, chewable formulations of the invention comprising less than about 70% (w/w) sugar, for example, less than about 60% (w/w), for example, less than about 50% (w/w), for example, less than about 40% (w/w), for example, less than about 30% (w/w) for example, less than about 25% sugar, such as less than about 20% (w/w), less than about 15% sugar, less than about 10%, less than about 5% or about 0% sugar.

In one embodiment, formulations of the invention further comprise an additive selected from sweeteners, food acids, flavouring agents, colouring agents, humectants, bulking agents, fatty acids, triglycerides, plasticizers, emulsifiers, thickeners, preservatives, and/or a mixture thereof. In a further embodiment, formulations of the invention comprise and additive selected from: sweeteners, food acids, flavouring agents, and colouring agents.

### Sweeteners

In general, an effective amount of sweetener may be utilized to provide the level of sweetness desired, and this amount may vary with the sweetener selected. Sweeteners may include one or more monosaccharides or disaccharides. Examples include sugar, sucrose, invert sugar, dextrose, lactose, honey, malt syrup, malt syrup solids, maltose, fructose, granular fructose, maple syrup, rice syrup, rice syrup solids, sorghum syrup, refiners syrup, corn syrup, corn syrup solids, high fructose corn syrup, molasses, or combinations thereof.

In one embodiment, the sweetener includes common sugars such as sucrose and glucose, polyols such as maltitol, erythritol, and isomalt, syrup sweeteners such as glucose syrup, corn syrup, high fructose corn syrup, and juice concentrates. In a further embodiment, the sweetener includes allulose.

In one embodiment, artificial sweeteners can be used such as acesulfame K, aspartame, sucralose, d-tagatose, neotame, monatin, and acesulfame potassium (Ace-K), or combinations thereof.

In a further embodiment sweeteners include saccharin, sodium saccharin, sodium cyclamate, acesulfame potassium, thaumatin, neohesperidin dihydrochalcone, ammonium glycyrrhizinate and aspartame.

The sweeteners involved may be selected from a wide range of materials including water-soluble sweeteners, water-soluble artificial sweeteners, water-soluble sweeteners derived from naturally occurring water-soluble sweeteners, dipeptide based sweeteners, and protein based sweeteners, including mixtures thereof. Without being limited to particular sweeteners, representative categories and examples include:
(a) water-soluble sweetening agents such as dihydrochalcones, monellin, steviosides, lo han quo, lo han quo derivatives, glycyrrhizin, dihydroflavenol, and sugar alcohols such as sorbitol, mannitol, maltitol, xylitol, erythritol, and L-aminodicarboxylic acid aminoalkenoic acid ester amides, such as those disclosed in U.S. Pat. No. 4,619,834, which disclosure is incorporated herein by reference, and mixtures thereof;
b) water-soluble artificial sweeteners such as soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfame-K), the free acid form of saccharin, and mixtures thereof;
(c) dipeptide based sweeteners, such as L-aspartic acid derived sweeteners, such as L-aspartyl-L-phenylalanine methyl ester (Aspartame), N—[NI-(3,3-dimethylbutyl)-L-.alpha.-aspartyl]-L-phenylalanine 1-methyl ester (Neotame), and materials described in U.S. Pat. No. 3,492,131, L-alphaaspartyl-N-(2,2,4,4-tetramethyl-3-thietanyl)-D-alaninamide hydrate (Alitame), methyl esters of L-aspartyl-L-phenylglycerine and L-aspartyl-L-2,5-dihydrophenyl-glycine, L-aspartyl-2,5-dihydro-L-phenylalanine; L-aspartyl-L-(I-cyclohexen)-alanine, and mixtures thereof;
(d) water-soluble sweeteners derived from naturally occurring water-soluble sweeteners, such as chlorinated derivatives of ordinary sugar (sucrose), e.g., chiorodeoxysugar derivatives such as derivatives of chlorodeoxysucrose or chlorodeoxygalactosucrose, known, for example, under the product designation of Sucralose; examples of chlorodeoxysucrose and chlorodeoxygalactosucrose derivatives include but are not limited to: 1-chloro-I′-deoxysucrose; 4-chloro-4-deoxy-alpha-D-galactopyranosyl-alpha-D-fructofuranoside, or 4-chloro-4-deoxygalactosucrose; 4-chloro-4-deoxy-alpha-D-galactopyranosyl-I-chloro-I-deoxy-beta-D-fructo-furanoside, or 4,r-dichloro-4,r-dideoxygalactosucrose; I′,6′-dichloro I′,6′-dideoxysucrose; 4-chloro-4-deoxy-alpha-D-galactopyranosyl-I,6-dichloro-I,6-dideoxy-beta-D-fructofuranoside, or 4,r,6′-trichloro-4,r,6′-trideoxygalactosucrose; 4,6-dichloro-4,6-dideoxy-alpha-D-galactopyranosyl-6-chloro-6-deoxy-beta-D-fructofuranoside, or 4,6,6′-trichloro-4,6,6′-trideoxygalactosucrose; 6,r,6′-trichloro-6,r,6′-trideoxysucrose; 4,6-dichloro-4,6-dideoxy-alpha-D-galacto-pyranosyl-I,6-dichloro-I,6-dideo-xy-beta-D-fructofuranoside, or 4,6,r,6′-tetrachloro-4,6, r,6′-tetradeoxygalacto-sucrose; and 4,6, r,6′-tetradeoxy-sucrose, and mixtures thereof;
(e) protein based sweeteners such as thaumaoccous danielli (Thaumatin I and II) and talin; and
(f) the sweetener monatin (2-hydroxy-2-(indol-3-ylmethyl)-4-aminoglutaric acid) and its derivatives. The intense sweetening agents may be used in many distinct physical forms well-known in the art to provide an initial burst of sweetness and/or a prolonged sensation of sweetness. Without being limited thereto, such physical forms include free forms, spray dried forms, powdered forms, beaded forms, encapsulated forms, and mixtures thereof. In one embodiment, the sweetener is a high intensity sweetener such as aspartame, sucralose, and acesulfame potassium (e.g., Ace-K or acesulfame-K).

In some embodiments, the sweetener may be a polyol. Polyols can include, but are not limited to glycerol, sorbitol, maltitol, maltitol syrup, mannitol, isomalt, erythritol, xylitol, hydrogenated starch hydrolysates, polyglycitol syrups, polyglycitol powders, lactitol, and combinations thereof.

In general, an effective amount of intense sweetener may be utilized to provide the level of sweetness desired, and this amount may vary with the sweetener selected. The intense sweetener may be present in amounts from about 0.001% to about 3%, by weight of the formulation, depending upon the sweetener or combination of sweeteners used. The exact range of amounts for each type of sweetener may be selected by those skilled in the art.

In one embodiment, the formulation may include sweeteners including, for example, sugar, glucose syrup, corn syrup, high fructose corn syrup, juice concentrate, or mixtures thereof. In one embodiment, the sweetener comprises erythritol, xylitol, sugar, glucose syrup, corn syrup, high fructose corn syrup, juice concentrate, tapioca syrup, agave syrup, brown rice syrup, high maltose syrup, invert sugar, trehalose, artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N—[N-[3-(3-hydroxy-4-methoxyphenyl)propyl]-L-.alpha.-aspartyl]-L-phenylalanine 1-methyl ester, N—[N-[3-(3-hydroxy-4-methoxyphenyl)-3-methylbutyl]-L-alpha-aspartyl]-L-phenylalanine 1-methyl ester, N—[N-[3-(3-methoxy-4-hydroxyphenyl)propyl]-L-alpha-aspartyl]-L-phenylalanine 1-methyl ester, salts thereof, liquorice or its extracts or isolates, or a mixture thereof.

### Food Acids

The pH of the formulation is about 2.5 to about 4, for example, about 2.7 to about 4, about 2.8 to about 3.7. The pH may be adjusted by a food acid, buffer, or both.

Suitable food acids include but are not limited to acetic acid, adipic acid, ascorbic acid, butyric acid, citric acid, formic acid, fumaric acid, lactic acid, phosphoric acid, malic acid, oxalic acid, succinic acid, tartaric acid, or combinations thereof.

Suitable buffers include but are not limited to: Sodium ascorbate, sodium citrate, sodium malate, sodium fumarate, potassium sodium tartrate and/or sodium tartrate.

### Flavouring Agents

In some embodiments, the formulation may further include a flavouring agent. Flavouring agents may include those flavours known to the skilled artisan, such as natural and artificial flavours. These flavourings may be chosen from synthetic flavour oils and flavouring aromatics and/or oils, oleoresins and extracts derived from plants, leaves, flowers, fruits, and so forth, and combinations thereof.

In some embodiments, the flavouring agents may include mint(s), menthol, menthone, isomenthone, camphor and eucalyptol, eucalyptol, camphor, borneol, fenchone, menthone and isomenthone, isopulegol, monomenthyl succinate, and menthyl lactate, menthone, isomenthone, borneol, fenchone, eucalyptus, ducalyptol, ethyl benzoate, neomenthol, d-fenchone, furfurylidene butyrate, bucchu fractions, sage oil, corn mint oil, rosemary, monomenthyl succinate, amyl salicylate, eugenol, phellendrene, propyl furoate, ethyl-3-hydroxy butyrate, hexyl valerate, anisyl propionate, anysyl butyrate, dihydrocarveol, or clary sag, Non-limiting representative flavour oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also useful flavourings are artificial, natural and synthetic fruit flavours such as vanilla, and citrus oils including lemon, orange, lime, grapefruit, yazu, sudachi, and fruit essences including apple, pear, peach, grape, blueberry, strawberry, raspberry, cherry, plum, pineapple, apricot, banana, melon, apricot, ume, cherry, raspberry, blackberry, tropical fruit, mango, mangosteen, pomegranate, papaya and so forth. Other potential flavours whose release profiles can be managed include a milk flavour, a butter flavour, a cheese flavour, a cream flavour, and a yogurt flavour; a vanilla flavour; tea or coffee flavours, such as a green tea flavour, a oolong tea flavour, a tea flavour, a cocoa flavour, a chocolate flavour, and a coffee flavour; mint flavours, such as a peppermint flavour, a spearmint flavour, and a Japanese mint flavour; spicy flavours, such as an asafetida flavour, an ajowan flavour, an anise flavour, an angelica flavour, a fennel flavour, an allspice flavour, a cinnamon flavour, a camomile flavour, a mustard flavour, a cardamom flavour, a caraway flavour, a cumin flavour, a clove flavour, a pepper flavour, a coriander flavour, a sassafras flavour, a savoury flavour, a Zanthoxyli Fructus flavour, a perilla flavour, a juniper berry flavour, a ginger flavour, a star anise flavour, a horseradish flavour, a thyme flavour, a tarragon flavour, a dill flavour, a capsicum flavour, a nutmeg flavour, a basil flavour, a marjoram flavour, a rosemary flavour, a bay leaf flavour, and a wasabi (Japanese horseradish) flavour; alcoholic flavours, such as a wine flavour, a whisky flavour, a brandy flavour, a rum flavour, a gin flavour, and a liqueur flavour; floral flavours; and vegetable flavours, such as an onion flavour, a garlic flavour, a cabbage flavour, a carrot flavour, a celery flavour, mushroom flavour, and a tomato flavour. These flavouring agents may be used in liquid or solid form and may be used individually or in admixture. Commonly used flavours include mints such as peppermint, menthol, spearmint, artificial vanilla, cinnamon derivatives, and various fruit flavours, whether employed individually or in admixture. Flavours may also provide breath freshening properties, particularly the mint flavours when used in combination with the cooling agents, described herein below. In some embodiments, flavourants may be chosen from geraniol, linalool, nerol, nerolidal, citronellol, heliotropine, methyl cyclopentelone, ethyl vanillin, maltol, ethyl maltol, furaneol, alliaceous compounds, rose type compounds such as phenethanol, phenylacetic acid, nerol, linalyl esters, jasmine, sandlewood, patchouli, and/or cedarwood.

In some embodiments, other flavourings include aldehydes and esters such as cinnamyl acetate, cinnamaldehyde, citral diethylacetal, dihydrocarvyl acetate, eugenyl formate, p-methylamisol, and so forth may be used. Generally, any flavouring or food additive such as those described in Chemicals Used in Food Processing, publication 1274, pages 63-258, by the National Academy of Sciences, may be used. This publication is incorporated herein by reference. These may include natural as well as synthetic flavours.

Further examples of aldehyde flavourings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, e.g., melonal (melon), 2,6-dimethyloctanal (green fruit), and 2-dodecenal (citrus, mandarin), cherry, grape, blueberry, blackberry, strawberry shortcake, and mixtures thereof.

In one embodiment, the flavouring agent comprises vanilla, peppermint oil, spearmint oil, eucalyptus oil, cinnamon oil, grapefruit oil, menthol, mono-menthyl succinate, menthol ethylene glycol carbonate, menthone glycerol ketal, menthyl lactate, (−)-isopulegol, p-menthane-3,8-diols, (−)-monomenthyl glutarate, oil of wintergreen (methylsalicy late), citrus oils, orange oils, fruit essences, Rosemary Oil, lavender oil, sage oil, clary sage oil, thyme oil, sandalwood oil, basil oil, coriander oil, cypress oil, fleabane oil, frankincense oil, geranium oil, fennel oil, oregano oil, Dalmatian sage oil, tarragon oil, or mixtures or derivatives thereof.

### Colouring Agents

Colouring agents may be used in amounts effective to produce the desired colour. The colouring agents may include pigments which may be incorporated in amounts up to about 6%, by weight of the formulation. The colourants may also include natural food colours and dyes suitable for food, drug and cosmetic applications. These colourants are known as F.D.& C. dyes and lakes. The materials acceptable for the foregoing uses are preferably water-soluble.

Illustrative nonlimiting examples include the indigoid dye known as F.D.& C. Blue No. 2, which is the disodium salt of 5,5-indigotindisulfonic acid. Similarly, the dye known as F.D.& C. Green No. 1 comprises a triphenylmethane dye and is the monosodium salt of 4-[4-(N-ethyl-p-sulfoniumbenzylamino) diphenylmethylene]-[I—(N-ethyl-N-p-sulfoniumbenzyl)-delta-2,5-cyclohexadieneimine], A full recitation of all F.D.& C. colourants and their corresponding chemical structures may be found in the Kirk-Othmer Encyclopaedia of Chemical Technology, 3rd Edition, in volume 5 at pages 857-884, which text is incorporated herein by reference.

In some embodiments, one or more colours can be included. As classified by the United States Food, Drug, and Cosmetic Act (21 C.F.R. 73), colours can include exempt from certification colours (sometimes referred to as natural even though they can be synthetically manufactured) and certified colours (sometimes referred to as artificial), or combinations thereof. In some embodiments, exempt from certification or natural colours can include, but are not limited to annatto extract, (EI60b), bixin, norbixin, astaxanthin, dehydrated beets (beet powder), beetroot red/betanin (E162), ultramarine blue, canthaxanthin (E161g), cryptoxanthin (E161c), rubixanthin (E161d), violanxanthin (E161e), rhodoxanthin (E161f), caramel (El50(a-d)), .beta.-apo-8′-carotenal (E160e), .beta.-carotene (E160a), alpha carotene, gamma carotene, ethyl ester of beta-apo-8 carotenal (E160f), flavoxanthin (E161a), lutein (E161b), cochineal extract (E120); carmine (E132), carmoisine/azorubine (E122), sodium copper chlorophyllin (E141), chlorophyll (E140), toasted partially defatted cooked cottonseed flour, ferrous gluconate, ferrous lactate, grape colour extract, grape skin extract (enocianina), anthocyanins (E163), haematococcus algae meal, synthetic iron oxide, iron oxides and hydroxides (E172), fruit juice, vegetable juice, dried algae meal, tagetes (Aztec marigold) meal and extract, carrot oil, corn endosperm oil, paprika, paprika oleoresin, phaffia yeast, riboflavin (E101), saffron, titanium dioxide, turmeric (E100), turmeric oleoresin, amaranth (E123), capsanthin/capsorbin (E160c), lycopene (E160d), and combinations thereof.

In some embodiments, certified colours can include, but are not limited to, FD&C blue #1, FD&C blue #2, FD&C green #3, FD&C red #3, FD&C red #40, FD&C yellow #5 and FD&C yellow #6, tartrazine (E102), quinoline yellow (E104), sunset yellow (E110), ponceau (E124), erythrosine (E127), patent blue V (E131), titanium dioxide (E171), aluminium (E173), silver (E174), gold (E175), pigment rubine/lithol rubine BK (E180), calcium carbonate (E170), carbon black (E153), black PN/brilliant black BN (E151), green S/acid brilliant green BS (E142), and combinations thereof. In some embodiments, certified colours can include FD&C aluminium lakes. These include of the aluminium salts of FD&C dyes extended on an insoluble substrate of alumina hydrate. Additionally, in some embodiments, certified colours can be included as calcium salts.

In some embodiments, natural fruits or plant juice or extracts may be used as the colouring agents. Example include without limitation carrot juice, raspberry juice, blackberry juice, blueberry juice, and beet juice.

### Humectant

The glycerine is a humectant and freezing point depressant. It also helps decrease the tendency for granulation and aid in maintaining softness. In some embodiments, glycerine or equivalent material may be employed at a level of from about 1 to about 5% by weight of the final product, e.g., 2 to 3%. In this specification, the term glycerine and glycerol are used interchangeably.

Humectants that can provide a perception of mouth hydration may be included. Such humectants can include, but are not limited to glycerol, sorbitol, polyethylene glycol, rythritol, and xylitol. Additionally, in some embodiments, fats can provide a perception of mouth moistening. Such fats can include medium chain triglycerides, vegetable oils, fish oils, mineral oils, and combinations thereof.

According to a further aspect of the invention there is provided a chewable formulation comprising:
a) a gelling component, for example, pectin, such as amidated low-methoxy pectin;
b) a compound of formula (I) or a salt thereof:

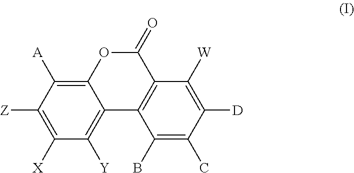

wherein:
A, B, C and D are each independently selected from H and OH;
W, X and Y are each independently selected from H and OH; and
Z is selected from H and OH; and
c) glycerol.

According to a further aspect of the invention there is provided a chewable formulation comprising:
a) a gelling component, for example, pectin, such as amidated low-methoxy pectin;
b) urolithin A, or a salt thereof, as defined above; and
c) glycerol.

According to a further aspect of the invention there is provided a formulation comprising:
a) pectin, such as amidated low-methoxy pectin;
b) a compound of formula (I) or a salt thereof:

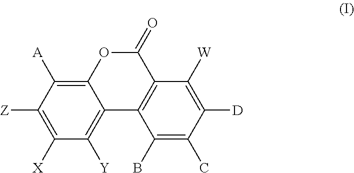

wherein:
A, B, C and D are each independently selected from H and OH;
W, X and Y are each independently selected from H and OH; and
Z is selected from H and OH; and
c) glycerol.

In some embodiments, glycerol may be employed at a level of from about 1 to about 10% by weight of the final product, for example, between about 1 and about 8% by weight of the final product, such as between about 2% and about 6%, between about 2% and about 3%, between about 2% and about 4%, between about 2% and about 6%, between about 3% and about 5%, between about 6% and about 8% of by weight of the final product. For example, about 1%, about 2%, about 3%, about 4%, about 5%, about 6%, about 7% or about 8% by weight of the final product.

According to a further aspect of the invention there is provided a chewable formulation comprising:
a) about 0.5% (w/w) to about 5% (w/w) pectin, for example, about 1.25% (w/w) to 3% (w/w) pectin, for example, amidated low-methoxy pectin;
b) about 1% (w/w) to about 20% (w/w) of a compound of formula (I) or a salt thereof, as defined above; for example, urolithin A;
c) about 0% (w/w) to about 10% (w/w) glycerol, for example about 2% (w/w) to about 6% (w/w) glycerol; and
d) about 20% (w/w) to about 75% (w/w) soluble fibre (for example, tapioca and/or Frutalose®).

According to a further aspect of the invention there is provided a chewable formulation comprising:
a) about 0.5% (w/w) to about 5% (w/w) pectin, for example, about 1.25% (w/w) to 3% (w/w) pectin, for example, amidated low-methoxy pectin;
b) about 1% (w/w) to about 20% (w/w) of a compound of formula (I) or a salt thereof, as defined above; for example, urolithin A;
c) about 0% (w/w) to about 10% (w/w) glycerol; for example about 2% (w/w) to about 6% (w/w) glycerol
d) about 20% (w/w) to about 75% (w/w) soluble fibre (for example, tapioca and/or Frutalose®); and
e) about 0.1% to 1% of one or more acids, for example, one or more acids selected from citric acid or malic acid.

According to a further aspect of the invention there is provided a chewable formulation comprising:
a) about 0.5% (w/w) to about 5% (w/w) pectin, for example, about 1.25% (w/w) to 3% (w/w) pectin, for example, amidated low-methoxy pectin;
b) about 1% (w/w) to about 20% (w/w) of a compound of formula (I) or a salt thereof, as defined above; for example, urolithin A;
c) about 0% (w/w) to about 10% (w/w) glycerol, for example about 2% (w/w) to about 6% (w/w) glycerol;
d) about 20% (w/w) to about 75% (w/w) soluble fibre (for example, tapioca and/or Frutalose®); and
e) about 0.1% to 1% of one or more acids, for example, one or more acids selected from citric acid or malic acid; and
f) a buffer.

According to a further aspect of the invention there is provided a formulation comprising:
a) about 0.5% (w/w) to about 5% (w/w) pectin, for example, about 1.25% (w/w) to 3% (w/w) pectin, for example, amidated low-methoxy pectin;
b) about 1% (w/w) to about 20% (w/w) of a compound of formula (I) or a salt thereof, as defined above; for example, urolithin A;
c) about 0% (w/w) to about 10% (w/w) glycerol, for example about 2% (w/w) to about 6% (w/w) glycerol; and
d) about 20% (w/w) to about 75% (w/w) soluble fibre (for example, tapioca and/or Frutalose®).

According to a further aspect of the invention there is provided a formulation comprising:
a) about 0.5% (w/w) to about 5% (w/w) pectin, for example, about 1.25% (w/w) to 3% (w/w) pectin, for example, amidated low-methoxy pectin;
b) about 1% (w/w) to about 20% (w/w) of a compound of formula (I) or a salt thereof, as defined above; for example, urolithin A;
c) about 0% (w/w) to about 10% (w/w) glycerol; for example about 2% (w/w) to about 6% (w/w) glycerol
d) about 20% (w/w) to about 75% (w/w) soluble fibre (for example, tapioca and/or Frutalose®); and
e) about 0.1% to 1% of one or more acids, for example, one or more acids selected from citric acid or malic acid.

According to a further aspect of the invention there is provided a formulation comprising:
a) about 0.5% (w/w) to about 5% (w/w) pectin, for example, about 1.25% (w/w) to 3% (w/w) pectin, for example, amidated low-methoxy pectin;
b) about 1% (w/w) to about 20% (w/w) of a compound of formula (I) or a salt thereof, as defined above; for example, urolithin A;
c) about 0% (w/w) to about 10% (w/w) glycerol, for example about 2% (w/w) to about 6% (w/w) glycerol;
d) about 20% (w/w) to about 75% (w/w) soluble fibre (for example, tapioca and/or Frutalose®); and
e) about 0.1% to 1% of one or more acids, for example, one or more acids selected from citric acid or malic acid; and
f) a buffer.

### Bulking Agents

Suitable sugar bulking agents include monosaccharides, disaccharides and polysaccharides such as xylose, ribulose, glucose (dextrose), lactose, mannose, galactose, fructose (levulose), sucrose (sugar), maltose, invert sugar, partially hydrolyzed starch and corn syrup solids, and mixtures thereof.

Suitable sugar alcohol bulking agents include sorbitol, xylitol, mannitol, galactitol, lactitol, maltitol, erythritol, isomalt and mixtures thereof. Suitable hydrogenated starch hydrolysates include those disclosed in U.S. Pat. No. 4,279,931 and various hydrogenated glucose syrups and/or powders which contain sorbitol, maltitol, hydrogenated disaccharides, hydrogenated higher polysaccharides, or mixtures thereof. Hydrogenated starch hydrolysates are primarily prepared by the controlled catalytic hydrogenation of corn syrups. The resulting hydrogenate starch hydrolysates are mixtures of monomeric, dimeric, and polymeric saccharides. The ratios of these different saccharides give different hydrogenated starch hydrolysates different properties. Mixtures of hydrogenated starch hydrolysates, such as LYCASIN®, a commercially available product manufactured by Roquette Freres of France, and HYSTAR®, a commercially available product manufactured by SPI Polyols, Inc. of New Castle, Del., are also useful.

In one embodiment, the bulking agent comprises maltitol syrup, polydextrose, sorbitol, soluble corn fibre, resistant starch, resistant maltodextrin, cellulose, hemicellulose, fructooligosaccharides, galacto-oligosaccharides, lactulose, xylo-isomalto-oligosaccharide, soybean oligosaccharide, oligo-glucose, stachyose, lactosucrose, or a combination thereof.

### Plasticizer

In some embodiments, the formulation may further include plasticizer to modify the texture of the formulation. A texture modifying agent may include a particulate material. Suitable particulate materials can include, but are not limited to, sucrose, polyols such as sorbitol, xylitol, mannitol, galactitol, lactitol, maltitol, erythritol, isomalt, hydrogenated starch hydrolysates and mixtures thereof, starches, proteins, and combinations thereof. In some embodiments, the particulate material serving as a texture modifying component is selected based on its ability or lack of ability to crystallize the saccharides in the saccharide portion. For example, when isomalt is included in the saccharide portion, sorbitol powder can be added to the formulation because it will not cause the isomalt to crystallize. Alternatively, when erythritol is included in the saccharide portion, erythritol powder can be added to the formulation because it will cause the erythritol to crystallize. Such particulates can be included in amounts from 5% to 35% w/w of the formulation.

### Emulsifiers

The formulation may include an emulsifier. The emulsifier may present in an amount of from about 0.001% to about 5%, alternatively 0.001% to 1%, alternatively 1% to 3%, alternatively 3% to 5%, by weight of the formulation. In some embodiments, the emulsifier is present in an amount of from about 0% to about 5%, alternatively 0.001% to 1%, alternatively 1% to 3%, alternatively 3% to 5%, by weight of the formulation.

Example emulsifiers include but not limited to modified corn starch, mono- and diglycerides, and lecithin.

The emulsifier may assist in holding together the fats and water and other components together in a homogeneous formulation. In one embodiment, the emulsifier may assist in the formation of a “water and oil” emulsion that creates the smooth texture of the finished product.

### Thickeners

The formulation may further include a thickening agent to help with the viscosity of the final product. Some thickening agents are gelling agents. Others act as mechanical thixotropic additives with discrete particles adhering or interlocking to resist strain.

In some embodiments, the thickening agent may be polysaccharides or protein. Example polysaccharides thickening agents include starches, vegetable gums and pectin. Example starch based thickening agents include arrowroot, cornstarch, katakuri starch, potato starch, sago, tapioca and their starch derivatives. Example vegetable gums based thickening agents may include alginin, guar gum, locust bean gum, and xanthan gum. Example protein based thickening agents include collagen, egg whites, furcellaran, and gelatin. Sugar based thickening agent may include agar and carrageenan

### Preservatives

Preservatives may be natural or synthetic. Non-limiting examples of suitable preservatives include: sodium benzoate, sodium citrate, sodium phosphate, potassium metabisulfite, sodium metabisulfite, sodium lactate, sodium sulfite, EDTA (ethylenediaminetetraacetic acid), methylparaben, TBHQ, tocopherols, and mixtures thereof. Natural preservatives may include phenols (phenolic acid, polyphenols, tannins), isoflavonoids, organic acids (acetic, lactic, citric), and herb extracts such as extracts of citrus fruits, oregano, thyme, sage, rosemary, clove, coriander, garlic, and onion.

In some embodiments, the formulation may include at least about 0% to 2%, by weight of the formulation of a preservative component from above, or mixtures thereof

### Gelling Components

The gelling component may include one or more gelling agents. A number of gelling agents may be utilized including without limitation, gelatin, pectin, gum Arabic, carrageenans, high methoxy pectin, alginates, gellan gum, modified or unmodified starches, modified starch wheat flour or enriched wheat flour or bleached flour or any type of flour from a natural source, or a combination thereof.

Other example gelling agents may include acacia, alginic acid, bentonite, Carbopols® (now known as carbomers), carboxymethyl cellulose, ethylcellulose, gelatin, hydroxyethyl cellulose, hydroxypropyl cellulose, magnesium aluminum silicate (Veegum®), methylcellulose, poloxamers (Pluronics®), polyvinyl alcohol, sodium alginate, and tragacanth.

The amount of gelling agents used in the formulation depend upon the texture, viscosity and softness of a desired product as well as other ingredients in the formulation In some embodiments, the gelling agents may be used in concentrations of about 0.5% to about 10%, about 0.1% to about 7%, or about 0.2 to about 15%.

In one embodiment, the gelatin and pectin may be employed at a weight ratio supplying at least 50% gelatin and at least 10% pectin, e.g., from about 70 to 85% gelatin and the remainder pectin.

In one embodiment, pectin may be a high methoxy pectin obtained from apples. In one embodiment, gelatin may be a type A gelatin from porcine sources. Bloom values for the gelatin may be in the range of from 100 to 280. In one embodiment, the bloom value is about 250.

In one embodiment, the combination of gelatin and pectin may be employed at a level of from about 4.5 to about 6% by weight of the final product, e.g., about 5.5% on that basis.

In one embodiment, the formulation may include gellan gum, carrageenans, or both providing a gelatin free formulation. In one embodiment, the formulation may include from about 0.25% to about 0.75% by weight gellan gum and about 2% to about 3% by weight carrageenan based on the total weight of the formulation.

In one embodiment, a combination of gellan gum at about 0.25% to about 0.75% by weight and carrageenans at about 2.5% to about 3% by weight based on total weight of the product produces a gummy formulation with TPA hardness values in excess of 20 lbs(f), and TPA cohesiveness and elasticity values of 75% to 80%.

In one embodiment, the amount of gellan gum is about 0.25 wt % to about 0.75 wt %, and about 0.25 wt % to about 0.5 wt %. In one embodiment, the amount of carrageenan is about 1.5 wt % to about 3 wt %, and about 2.5 wt % to about 3 wt %.

According to a further aspect of the invention there is provided a formulation comprising:
a) a compound of formula (I) or a salt thereof:

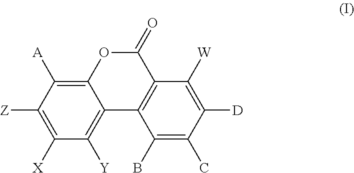

wherein:
A, B, C and D are each independently selected from H and OH;
W, X and Y are each independently selected from H and OH; and
Z is selected from H and OH; and
b) pectin, for example, high methoxy pectin.

According to a further aspect of the invention, there is provided a formulation, comprising;
(a) a compound of formula (I); for example, urolithin A, and
(b) pectin;
wherein the ratio of the compound of formula (I) and pectin is in the range about 1:2 (w/w) to about 10 to 1 (w/w).

According to a further aspect of the invention, there is provided a formulation, comprising;
(a) a compound of formula (I); for example, urolithin A,
(b) pectin; and
(c) soluble fibre, for example, soluble tapioca fibre.

According to a further aspect of the invention, there is provided a formulation, comprising;
(a) a compound of formula (I); for example, urolithin A,
(b) pectin; and
(c) a low calorie sweetener, for example, allulose.

According to a further aspect of the invention, there is provided a formulation, comprising;
(a) a compound of formula (I); for example, urolithin A,
(b) pectin; and
(c) citric acid and/or a citrate salt (for example, sodium citrate), for example, citric acid and a salt of citric acid (for example, sodium citrate).

According to a further aspect of the invention, there is provided a formulation, comprising;
(a) a compound of formula (I); for example, urolithin A,
(b) pectin;
(c) soluble tapioca fibre;
(d) citric acid; and/or a citrate salt (for example, sodium citrate); and
(e) a low calorie sweetener, for example, allulose.

According to a further aspect of the invention, there is provided a formulation, comprising;
(a) about 1% to about 20% (w/w), for example, about 5% to about 20% (w/w), compound of formula (I); for example, urolithin A, and
(b) about 0.5% to about 4% (w/w) pectin;

According to a further aspect of the invention, there is provided a formulation, comprising;
(a) about 1% to about 20% (w/w), for example, about 5% to about 20% (w/w), a compound of formula (I); for example, urolithin A,
(b) about 0.5% to about 4% (w/w) pectin; and
(c) about 25% to about 45% (w/w) soluble tapioca fibre.

According to a further aspect of the invention, there is provided a formulation, comprising;
(a) about 1% to about 20% (w/w), for example, about 5% to about 20% (w/w), compound of formula (I); for example, urolithin A,
(b) about 0.5% to about 4% (w/w) pectin; and
(c) about 25% to about 45% (w/w) low calorie sweetener, for example, allulose.

According to a further aspect of the invention, there is provided a formulation, comprising;
(a) about 1% to about 20% (w/w), for example, about 5% to about 20% (w/w), compound of formula (I); for example, urolithin A,
(b) about 0.5% to about 4% (w/w) pectin; and
(c) about 1% to about 10% (w/w), for example, about 1% to 5% or about 2% to about 4%, low calorie sweetener, for example, allulose.

According to a further aspect of the invention, there is provided a formulation, comprising;
(a) about 1% to about 20% (w/w), for example, about 5% to about 20% (w/w), compound of formula (I); for example, urolithin A,
(b) about 0.5% to about 4% (w/w) pectin; and
(c) about 25% to about 60% (w/w), low calorie sweetener, for example, one or more low calorie sweeteners selected from allulose, fructalose, stevia and/or inulin.

According to a further aspect of the invention, there is provided a formulation, comprising;
(a) about 1% to about 20% (w/w), for example, about 5% to about 20% (w/w), compound of formula (I); for example, urolithin A,
(b) about 0.5% to about 4% (w/w) pectin; and
(c) about 0.5% to about 2% (w/w) citric acid and/or a citrate salt (for example, sodium citrate), for example, citric acid and a salt of citric acid (for example, sodium citrate).

According to a further aspect of the invention, there is provided a formulation, comprising;
(a) about 1 to about 20% (w/w), for example, about 5% to about 20% (w/w), compound of formula (I); for example, urolithin A,
(b) about 0.5% to about 4% (w/w) pectin;
(c) about 25% to about 45% (w/w) soluble tapioca fibre;
(d) about 0.5% to about 2% (w/w) citric acid; and/or a citrate salt (for example, sodium citrate); and
(e) about 25% to about 45% (w/w) low calorie sweetener, for example, allulose.

Embodiments of the invention comprise compound of formula (I) (for example, urolithin A) in the range about 5% to about 20% (w/w). For example, between about 5% to about 15%, for example about 5% to about 10% (w/w).

Embodiments of the invention, comprise pectin (for example, high methoxy pectin) in the range of about 0.5% to about 10% (w/w), about 0.5% to about 5% (w/w), about 0.5% to about 4% (w/w), about 0.5% to about 3% (w/w), about 0.5% to about 2% (w/w) pectin. For example, about 0.5% to about 1.5% (w/w), about 0.8% to about 1.5% (w/w), for example, about 1% to about 1.5% (w/w), for example about 1% to about 4%, or about 2% to about 4% (w/w) pectin.

Embodiments of the invention comprise soluble tapioca fibre (for example, soluble tapioca fibre syrup) in the range of about 25% to about 45% (w/w). For example, about 30% to about 40%, about 32% to about 38% (w/w).

Embodiments of the invention comprise citric acid; and/or a citrate salt (for example, sodium citrate) in the range about 0.5% to about 2% (w/w). For example, about 0.8% to about 1.5%, about 1% to about 1.5% (w/w). Salts of citric acid include sodium citrate, potassium citrate, calcium citrate and tri-ammonium citrate.

In a further aspect of the invention, formulations comprise both citric acid and a salt of citric acid. Embodiments of the invention comprise citric acid in the range of about 0.5% to about 2% (w/w). For example, about 0.5% to about 1.5%, about 0.8% to about 1.5%, about 1% to about 1,5%, about 0.8% to about 1.3% (w/w). Embodiments of the invention comprise a salt of citric acid (for example, sodium citrate) in the range of about 0.1% to about 1% (w/w). For example, about 0.1% to about 0.8%, about 0.1% to about 0.5% (w/w).

In a further aspect of the invention, formulations comprise both malic acid and sodium hydrogen malate.

Embodiments of the invention comprise low calorie sweetener, (for example, allulose) in the range about 25% to about 45% (w/w). For example, about 30% to about 40%, about 32% to about 38% (w/w).

According to a further aspect of the invention, there is provided a formulation, comprising;
(a) about 1% to about 20% (w/w), for example, about 5% to about 20% (w/w), compound of formula (I); for example, urolithin A,
(b) about 0.5% to about 4% (w/w) pectin; and
(c) about 25% to about 60% (w/w), low calorie sweetener, for example, one or more low calorie sweeteners selected from allulose, fructalose, monk fruit juice, stevia and/or inulin.
(d) about 10% to about 30% soluble fibre;
(e) about 0.1% to about 2% sodium citrate, for example, about 0.3% to about 1% sodium citrate;
(f) optionally a pH buffer, for example, malic acid and/or sodium hydrogen malate; and
(g) optionally a colouring agent.

### Compounds of Formula (I) in Amorphous Form

Compounds of Formula (I) exist in crystalline form, however, we have found that compounds of Formula (I) in amorphous form are useful in the preparation of higher bioavailability compositions. Therefore, according to a further aspect of the invention, there is provided a compound of Formula (I) in amorphous form, for example, amorphous urolithin A.

In a further embodiment of the invention, there is provided a formulation of the invention, wherein the compound of Formula (I) is in amorphous form.

Furthermore, we have found that bioavailability can be enhanced by the addition of one or more excipients. Therefore, according to a further aspect of the invention there provided a composition comprising a compound of Formula (I), in amorphous form, for example, amorphous urolithin A and one or more excipients.

Excipients include one of more of the following: pectin, methylcellulose, hydroxypropyl methylcellulose (for example, HPMC E5), arabic gum, alginate (for example, sodium alginate, gelatin, mannitol and sorbitol. In a further embodiment excipients further include: agar, starch or modified starch.

In a further embodiment the invention provides a composition comprising a compound of Formula (I) in amorphous form and an excipient wherein the compound of Formula (I) and excipient(s) are in a ratio of 1 to about 1 to 1 to about 10, 1 to about 2 to 1 to about 10, a ratio of 1 to about 2 to 1 to about 9, a ratio of 1 to about 2 to 1 to about 8, a ratio of 1 to about 2 to 1 to about 7, a ratio of 1 to about 2 to 1 to about 6, a ratio of 1 to about 2 to 1 to about 5, for example, a ratio of 1 to about 2 to 1 to about 4, for example, a ratio of 1 to about 3, for example, wherein the excipient is selected from one or more of pectin, methylcellulose, hydroxypropyl methylcellulose (for example, HPMC E5), arabic gum, alginate and gelatin. In a further embodiment, the invention provides a composition comprising a compound of Formula (I) in amorphous form and an excipient wherein the excipient is mixture of pectin and methylcellulose.

In one embodiment, the invention comprises a compound of Formula (I) in amorphous form, for example, amorphous urolithin A, and hydroxypropyl methylcellulose (for example, HPMC E5) in a ratio of 1 to about 3.

In one embodiment, the invention comprises a compound of Formula (I) in amorphous form, for example, amorphous urolithin A, and methylcellulose in a ratio of 1 to about 3.

In one embodiment, the invention comprises a compound of Formula (I) in amorphous form, for example, amorphous urolithin A, and pectin in a ratio of 1 to about 3, ratio of 1 to about 4 or a ratio of 1 to about 5.

In one embodiment, the invention comprises a compound of Formula (I) in amorphous form, for example, amorphous urolithin A, and a mixture of excipient comprising pectin and methyl cellulose. In one embodiment the compound of Formula (I), pectin and methyl cellulose are in a ratio of 1 to about 3 to about 2.

According to a further aspect of the invention there is provided a formulation comprising:
(a) a compound of Formula (I), for example, urolithin A;
(b) pectin;
(c) a source of fibre; for example, one of more sources of fibre selected from tapioca, corn fibre and inulin;
(d) one or more sweeteners, for example, allulose and or stevia;
(e) water;
(f) citric acid and/or sodium citrate’
(g) optionally one or more colouring agents;
(h) optionally one or more flavouring agents.

According to a further aspect of the invention there is provided a formulation comprising:
(a) a compound of Formula (I), for example, urolithin A;
(b) pectin;
(c) a source of fibre; for example, tapioca;
(d) one or more sweeteners, for example, one or more sweeteners selected from allulose, stevia and monk fruit juice;
(e) a pH regulator, for example, sodium hydrogen mallate;
(f) sodium citrate;
(g) water;
(h) optionally one or more colouring agents;
(i) optionally one or more flavouring agents; and
(j) optionally a coating.

According to a further aspect of the invention there is provided a formulation comprising:
(a) a compound of Formula (I), for example, urolithin A;
(b) pectin;
(c) glycerol;
(d) a source of fibre; for example, one of more sources of fibre selected from tapioca, corn fibre and inulin;
(e) one or more sweeteners, for example, allulose and or stevia;
(f) water;
(g) citric acid and/or sodium citrate’
(h) optionally one or more colouring agents;
(i) optionally one or more flavouring agents.

According to a further aspect of the invention there is provided a formulation comprising:
(a) a compound of Formula (I), for example, urolithin A;
(b) pectin;
(c) glycerol;
(d) a source of fibre; for example, tapioca;
(e) one or more sweeteners, for example, one or more sweeteners selected from allulose, stevia and monk fruit juice;
(f) a pH regulator, for example, sodium hydrogen mallate;
(g) sodium citrate;
(h) water;
(i) optionally one or more colouring agents;
(j) optionally one or more flavouring agents; and
(k) optionally a coating.

According to a further aspect of the invention there is provided a formulation comprising:
(a) a compound of Formula (I), for example, urolithin A;
(b) Gum Arabic;
(c) optionally glycerol;
(d) a source of fibre; for example, one of more sources of fibre selected from tapioca, corn fibre and inulin;
(e) one or more sweeteners, for example, allulose and or stevia;
(f) water;
(g) citric acid and/or sodium citrate’
(h) optionally one or more colouring agents;
(i) optionally one or more flavouring agents.

According to a further aspect of the invention there is provided a formulation comprising:
(a) a compound of Formula (I), for example, urolithin A;
(b) Gum Arabic;
(c) optionally glycerol;
(d) a source of fibre; for example, tapioca;
(e) one or more sweeteners, for example, one or more sweeteners selected from allulose, stevia and monk fruit juice;
(f) a pH regulator, for example, sodium hydrogen mallate;
(g) sodium citrate;
(h) water;
(i) optionally one or more colouring agents;
(j) optionally one or more flavouring agents; and
(k) optionally a coating.

### Spray-Dried Compositions of Compounds of Formula(I)

It has been found that compositions can be prepared by dissolving a compound of Formula (I), for example, urolithin A, in a solvent and spray drying. Therefore, according to a further aspect of the invention, there is provided a spray dried composition of a compound of Formula (I), for example, urolithin A.

Solvents suitable for spray drying include: ethanol and water.

In one embodiment, the solvent is water.

In a further embodiment, the solvent is ethanol.

In one embodiment, the excipient is pectin, for example, low methoxy pectin.

In a further embodiment, the excipient is a mixture of pectin and methylcellulose.

In a further embodiment, the excipient is a methylcellulose.

In a further embodiment, the excipient is hydroxypropyl methylcellulose, for example HPMC E5.

In a further embodiment, the excipient is a gum Arabic.

In a further embodiment, the solvent is water and the excipient is pectin.

In a further embodiment, the solvent is ethanol and the excipient is pectin.

In a further embodiment of the invention, there is provided a spray dried composition of a compound of Formula (I), for example, urolithin A, comprising an excipient, for example, an excipient selected from one or more of pectin, for example, high methoxy-pectin, gum Arabic, melthylcellulose and hydroxypropyl-methylcellulose, for example, HPMC-E5, sorbitol, mannitol and sodium alginate.

In a further embodiment of the invention, there is provided a spray dried composition of a compound of Formula (I), for example, urolithin A, comprising pectin, for example, high methoxy-pectin.

In a further embodiment of the invention, there is provided a spray dried composition of a compound of Formula (I), for example, urolithin A, comprising methylcellulose.

In a further embodiment of the invention, there is provided a spray dried composition of a compound of Formula (I), for example, urolithin A, comprising gum Arabic.

In a further embodiment of the invention, there is provided a spray dried composition of a compound of Formula (I), for example, urolithin A, comprising hydroxypropyl methylcellulose, for example HPMC E5.

According to a further embodiment of the invention there is provided a process for preparing a composition comprising a compound of Formula (I), comprising:
(a) dissolving a compound of Formula (I) in a solvent to form a solution, or suspending a compound of Formula (I) in a solvent to form a suspension, for example, wherein the solvent is selected from ethanol and/or water;
(b) optionally stirring the resulting suspension or solution
(c) adding one or more excipients, for example, one or more excipients selected from pectin, methylcellulose, and hydroxypropyl methylcellulose;
(d) spray drying the solution/suspension, optionally with stirring, to form a powder; and
(e) optionally drying the powder.

According to a further embodiment of the invention, there is provided a composition preparable by a process comprising:
(a) dissolving a compound of Formula (I) in a solvent to form a solution, or suspending a compound of Formula (I) in a solvent to form a suspension, for example, wherein the solvent is selected from ethanol and/or water;
(b) optionally stirring the resulting suspension or solution
(c) adding one or more excipients, for example, one or more excipients selected from pectin, methylcellulose, and hydroxypropyl methylcellulose;
(d) spray drying the solution/suspension, optionally with stirring, to form a powder; and
(e) optionally drying the powder.

In a further embodiment of the invention, a spray dried composition comprising a compound of Formula (I) and a low methoxy pectin.

According to a further aspect of the invention there is provided a spray-dried formulation comprising:
a) pectin, for example, low-methoxy pectin, such as amidated low-methoxy amidated pectin; and
b) a compound of formula (I) or a salt thereof:

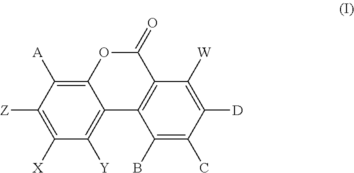

wherein:
A, B, C and D are each independently selected from H and OH;
W, X and Y are each independently selected from H and OH; and
Z is selected from H and OH.

According to a further aspect of the invention there is provided a spray-dried formulation comprising:
a) pectin, for example, low-methoxy pectin, such as amidated low-methoxy amidated pectin; and, and
b) urolithin A.

According to a further aspect of the invention there is provided a spray-dried formulation comprising:
a) pectin, for example, low-methoxy pectin, such as amidated low-methoxy amidated pectin; and
b) a compound of formula (I) or a salt thereof, as defined above;
wherein the ratio of a compound of formula (I) to pectin is about 1 to 1 (w/w).

According to a further aspect of the invention there is provided a spray-dried formulation comprising:
a) pectin, for example, low-methoxy pectin, such as amidated low-methoxy amidated pectin; and, and
b) urolithin A:
wherein the ratio of a compound of urolithin A to pectin is about 1 to 1 (w/w).

According to a further aspect of the invention there is provided a spray-dried formulation comprising:
a) gum Arabic, and
b) a compound of formula (I) or a salt thereof:

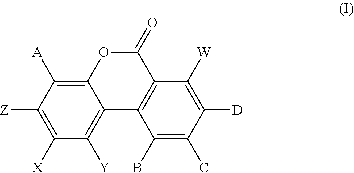

wherein:
A, B, C and D are each independently selected from H and OH;
W, X and Y are each independently selected from H and OH; and
Z is selected from H and OH.

According to a further aspect of the invention there is provided a spray-dried formulation comprising:
a) gum Arabic, and
b) urolithin A:

According to a further aspect of the invention there is provided a spray-dried formulation comprising:
a) gum Arabic; and
b) a compound of formula (I) or a salt thereof, as defined above;
wherein the ratio of a compound of formula (I) to Gum Arabic is about 1 to 1 (w/w).

According to a further aspect of the invention there is provided a spray-dried formulation comprising:
a) gum Arabic, and
b) urolithin A:
wherein the ratio of a compound of urolithin A to Gum Arabic is about 1 to 1 (w/w).

According to a further aspect of the invention there is provided a spray-dried formulation comprising:
a) alginate, and
b) a compound of formula (I) or a salt thereof:

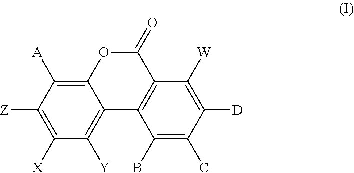

wherein:
A, B, C and D are each independently selected from H and OH;
W, X and Y are each independently selected from H and OH; and
Z is selected from H and OH.

According to a further aspect of the invention there is provided a spray-dried formulation comprising:
a) alginate, and
b) urolithin A:

According to a further aspect of the invention there is provided a spray-dried formulation comprising:
c) gum Arabic, and
d) urolithin A:

According to a further aspect of the invention there is provided a spray-dried formulation comprising:
a) alginate; and
b) a compound of formula (I) or a salt thereof, as defined above;
wherein the ratio of a compound of formula (I) to alginate is about 1 to 1 (w/w).

According to a further aspect of the invention there is provided a spray-dried formulation comprising:
a) alginate, and
b) urolithin A:
wherein the ratio of a compound of urolithin A to alginate is about 1 to 1 (w/w).

According to a further aspect of the invention there is provided a spray-dried formulation comprising:
a) cyclodextrin, and
b) a compound of formula (I) or a salt thereof:

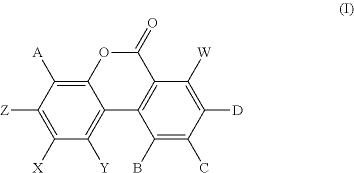

wherein:
A, B, C and D are each independently selected from H and OH;
W, X and Y are each independently selected from H and OH; and
Z is selected from H and OH.

According to a further aspect of the invention there is provided a spray-dried formulation comprising:
a) cyclodextrin, and
b) urolithin A:

According to a further aspect of the invention there is provided a spray-dried formulation comprising:
a) cyclodextrin; and
b) a compound of formula (I) or a salt thereof, as defined above;
wherein the ratio of a compound of formula (I) to cyclodextrin is about 1 to 1 (w/w).

According to a further aspect of the invention there is provided a spray-dried formulation comprising:
a) cyclodextrin, and
b) urolithin A:
wherein the ratio of a compound of urolithin A to cyclodextrin is about 1 to 1 (w/w).

According to a further aspect of the invention there is provided a spray-dried formulation comprising:
a) maltodextrin selected from digestible and digestion-resistant spray-dried, and
b) a compound of formula (I) or a salt thereof:

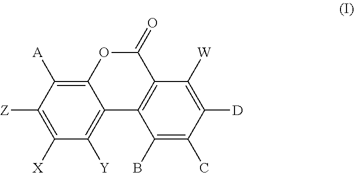

wherein:
A, B, C and D are each independently selected from H and OH;
W, X and Y are each independently selected from H and OH; and
Z is selected from H and OH.

According to a further aspect of the invention there is provided a spray-dried formulation comprising:
a) maltodextrin selected from digestible and digestion-resistant maltodextrin, and
b) urolithin A:

According to a further aspect of the invention there is provided a spray-dried formulation comprising:
a) maltodextrin selected from digestible and digestion-resistant maltodextrin; and
b) a compound of formula (I) or a salt thereof, as defined above;
wherein the ratio of a compound of formula (I) to maltodextrin is about 1 to 1 (w/w).

According to a further aspect of the invention there is provided a spray-dried formulation comprising:
a) maltodextrin selected from digestible and digestion-resistant maltodextrin, and
b) urolithin A:
wherein the ratio of a compound of urolithin A to maltodextrin is about 1 to 1 (w/w).

In further embodiments of spray-dried formulations of the invention as described above, the ratio of the compound of formula (I) or urolithin A to the pectin, gum Arabic, alginate, cyclodextrin or maltodextrin is about 1 to 3 to about 3 to 1 (w/w), for example, about 1 to about 2.5 (w/w) to about 2.5 to 1 (w/w), for example, about 1 to 2 to about 2 to 1 (w/w), for example, about 1 to 1.5 to about 1.5 to 1 (w/w).

In further embodiments of the invention the ratio of a compound of formula (I) or urolithin A and pectin, for example, low methoxy pectin such as amidated low methoxy pectin is about 20 to 1 (w/w) to about 1 to 30 (w/w), for example, between about 10 to 1 (w/w) to about 1 to 20 (w/w), about 10 to 1 (w/w) to about 1 to 10 (w/w), or about 5 to 1 (w/w) to about 1 to 5 (w/w).

In further embodiments of the invention the ratio of a compound of formula (I) or urolithin A and pectin, for example, low methoxy pectin such as amidated low methoxy pectin is about 10 to 1 (w/w) to about 1 to 1 (w/w), for example, about 8 to 1 (w/w) to about 1 to 1 (w/w), about 7 to 1 (w/w) to about 1 to 1 (w/w), about 5 to 1 (w/w) to about 1 to 1 (w/w), about 3 to 1 (w/w) to about 1 to 10 (w/w) or about 1 to 1 (w/w).

In further embodiments, the ratio of urolithin A to pectin is selected from one of the following:
Ratio about 1:1 (w/w), for example, UA 500 mg/Pectin 500 mg;
Ratio about 3:1 (w/w), for example, UA 500 mg/Pectin 167 mg;
Ratio about 4:1 (w/w), for example, UA 500 mg/Pectin 125 mg;
Ratio about 7:1 (w/w), for example, UA 500 mg/Pectin 71.4 mg;
Ratio about 10:1 (w/w), for example UA 500 mg/Pectin 50 mg;
Ratio about 1:30 (w/w), for example, UA 500 mg/Pectin 15 g;
Ratio about 1:10 (w/w), for example, UA 500 mg/Pectin 5 g;
Ratio about 1:4 (w/w), for example, UA 500 mg/Pectin 2000 mg; or
Ratio about 20:1 (w/w), for example UA 500 mg/Pectin 25 mg.

In a further embodiment a composition of the invention, comprises:

| | Range |
|---|---|
| Ingredients | (w/w) |
| Allulose | 25%-45% |
| Soluble Tapioca Fiber Syrup | 25%-45% |
| Water | 15%-25% |
| Urolithin A | 5% to 15% |
| Pectin | 2% to 10% |
| Citric Acid, | 1% to 2% |
| Stevia | 0.005%-0.010% |
| Sodium Citrate | 0.1% to 0.5% |

wherein the ranges add up to 100% with any optional colouring agents added.

In a further embodiment a composition of the invention, comprises:

| Raw material | Weight % (wet |
|---|---|
| Description | gummy slurry) |
| Allulose liquid | 1% to 5% |
| Fibersol Liquid 2 L | 10% to 30% |
| fructalose | 45% to 65% |
| Water | 1% to 3% |
| Urolithin A | 5% to 15% |
| Pectin | 1% to 4% |
| Sodium Hydrogen Malate | 0.1% to 0.4% |
| Monk Fruit Conc Juice | 0.1% to 1% |
| Stevia | 0.05% to 0.3% |
| Sodium Citrate | 0.1% to 1% |
| Malic Acid | 0.1% to 1% |

wherein the ranges add up to 100% with any optional colouring agents added.

### Urolithins

Urolithins are metabolites produced by the action of mammalian, including human, gut microbiota on ellagitannins and ellagic acid. Ellagitannins and ellagic acid are compounds commonly found in foods such as pomegranates, nuts and berries. Ellagitannins are minimally absorbed in the gut themselves. Urolithins are a class of compounds with the representative structure (I) shown below. The structures of some particularly common urolithins are described in the Table below, with reference to structure (I).

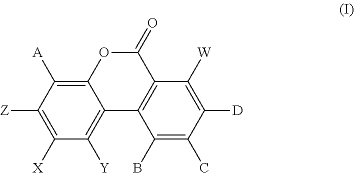

Substituent of structure (I)

| | A | B | C | D | W, X and Y | Z |
|---|---|---|---|---|---|---|
| Urolithin A | H | H | H | OH | H | OH |
| Urolithin B | H | H | H | H | H | OH |
| Urolithin C | H | H | OH | OH | H | OH |
| Urolithin D | OH | H | OH | OH | H | OH |
| Urolithin E | OH | OH | H | OH | H | OH |
| Isourolithin A | H | H | OH | H | H | OH |
| Isourolithin B | H | H | OH | H | H | H |
| Urolithin M-5 | OH | OH | OH | OH | H | OH |
| Urolithin M-6 | H | OH | OH | OH | H | OH |
| Urolithin M-7 | H | OH | H | OH | H | OH |

In practice, for commercial scale products, it is convenient to synthesise the urolithins. Routes of synthesis are described, for example, in WO 2014/004902, WO 2015/100213 and WO 2019/168972.

Urolithins of any structure according to structure (1) may be used in the combinations of the invention.

In one aspect of a combinations of the invention, a suitable compound is a compound of formula (I) wherein A, C, D and Z are independently selected from H and OH and B, W, X and Y are all H, preferably at least one of A, C, D and Z is OH.

Particularly suitable compounds are the naturally-occurring urolithins. Thus, Z is preferably OH and W, X and Y are preferably all H. When W, X and Y are all H, and A, and B are both H, and C, D and Z are all OH, then the compound is Urolithin C. When W, X and Y are all H, and A, B and C are all H, and D and Z are both OH, then the compound is urolithin A. Preferably, the urolithin used in the methods of the present disclosure is urolithin A, urolithin B, urolithin C or urolithin D. Most preferably, the urolithin used is urolithin A.

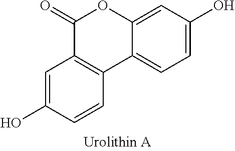 [SMILES: O=c1oc2cc(O)ccc2c2ccc(O)cc12]

According to one embodiment there is provided a combination of the invention wherein the compound of formula (I) is urolithin A.

According to one embodiment there is provided a combination of the invention wherein the compound of formula (I) is urolithin B.

According to one embodiment there is provided a combination of the invention wherein the compound of formula (I) is urolithin C.

According to one embodiment there is provided a combination of the invention wherein the compound of formula (I) is urolithin D.

In one embodiment, urolithins do not include acylated urolithins or optionally substituted acylated urolithins, (for example, acylated urolithin A, acylated urolithin B, acylated urolithin C, acylated urolithin D, acylated urolithin E, or acylated urolithin M5; orurolithin C having at least one hydroxyl substituted with a group containing a fatty acid). The term “acyl,” as used herein, represents a chemical substituent of formula —C(O)—R, where R is alkyl, alkenyl, aryl, arylalkyl, cycloalkyl, heterocyclyl, heterocyclyl alkyl, heteroaryl, or heteroaryl alkyl. An optionally substituted acyl is an acyl that is optionally substituted as described herein for each group R. Examples of acyl include fatty acid acyls (e.g., short chain fatty acid acyls (e.g., acetyl)) and benzoyl.

The present invention also encompasses use of suitable salts of compounds of formula (I), e.g. pharmaceutically acceptable salts. Suitable salts according to the invention include those formed with organic or inorganic bases. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine.

### Additional Components in Formulations of the Invention:

The formulations according to the invention may contain additional components beyond the urolithin and the gelling component. The additional components may be compounds that provide health benefits, for example, a mineral formulation, an antioxidant formulation, or a mitochondria boosting formulation.

In one embodiment, the antioxidant formulation comprises bioflavonoids, resveratrol, coenzyme Q10, quercetin, rutin, lycopene, L-glutathione, N-acetyl cysteine, phenolics, anthocyanins, flavonoids, anthracenes, carotenoids, zeaxanthin, astaxanthin, xanthin, pomegranate, Ginkgo biloba, green tea, garlic, grapeseed, blackberry, elderberry, cranberry, blueberry, saffron, Sangre de grado (dragon's blood), lyceum barbarum (Gouqizi), its extract, powder, or isolates thereof.

In one embodiment, the mitochondria boosting formulation comprises acetyl L-carnitine, alpha-lipoic acid, coenzyme Q10 (CoQIO, or ubiquinone), nicotinamide riboside (NR), omega-3 fatty acids, magnesium, D-ribose, or a derivative or combination thereof

Creatine has been described as having beneficial effects in the treatment of muscle disorders. It can be included in formulation of the invention. β-hydroxyl-β-methylbutyrate (HMB) has been described as having beneficial effects in the treatment of muscle disorders. It can be included in formulation of the invention.

In a further embodiment the formulation further comprises spermidine.

Examples of mineral formulations include potassium, chromium pocolinate, magnesium and selenium. In one embodiment, the mineral formulation may include ions of sodium, magnesium, chromium, iodine, iron, manganese, calcium, copper, fluoride, potassium, phosphorous, molybdenum, selenium, zinc, and combinations thereof. The minerals may be in the forms of salts or chelates.

### Therapeutic Uses

### Inflammatory Diseases, Disorders and Conditions

According to a further embodiment of the invention, there is provided a formulation of the invention for inhibiting inflammation. According to a further embodiment of the invention, there is provided a formulation of the invention for relieving or treating acute inflammation or chronic inflammation-induced diseases. In one embodiment, the acute inflammation is, for example, influenza virus pneumonia, new coronavirus pneumonia, and acute lung injury inflammation. In a further embodiment, the chronic inflammation is, for example, chronic obstructive pneumonia, chronic asthmatic airway inflammation and chronic obstructive pulmonary disease (COPD).

According to a further embodiment of the invention, there is provided a formulation of the invention, for treating or preventing an inflammatory disease, for example, an inflammatory disease selected from inflammatory skin diseases, Crohn's disease, ulcerative colitis, peritonitis, osteomyelitis, meningitis, meningitis, encephalitis, pancreatitis, trauma-induced shock, bronchial asthma, allergic rhinitis, cystic fibrosis, Inflammatory bowel disease, acute bronchitis, chronic bronchitis, osteoarthritis, gout, spondyloarthropathies, ankylosing spondylitis, intestinal spondylitis, inflammatory arthropathy, ankylosing spondylitis, reactive arthropathy, infectious arthritis, systemic lupus erythematosus, recurrent and psoriasis. For example, an inflammatory disease selected from arthritis, gonococcal arthritis, tuberculous arthritis, viral arthritis, fungal arthritis, rheumatoid arthritis, rheumatoid polyposis muscle pain, arthritic arthritis, calcium arthritis, non-articular rheumatism, bursitis, and hay fever.

### Infectious Diseases

According to one embodiment of the invention, there is provided a formulation of the invention for use in the treatment of microbial diseases. In a further embodiment, there is provided a formulation of the invention for use in the treatment of viral diseases.

According to a future embodiment of the invention, there is provided a formulation of the invention, for use in treating a post-viral illness, for example, symptoms associated with recovery from the viral disease or infection in the subject. Symptoms associated with recovery from a viral disease comprise fatigue, post-exertional malaise (PEM), problems with memory or concentration, sore throat, headache, muscle or joint pain, dizziness, brain fog, shortness of breath, and/or unrefreshing sleep. In one embodiment, the post-viral illness is selected from myalgic encephalomyelitis/chronic fatigue syndrome, long-COVID (for example, caused by an infection with a delta variant or an omicron variant of SARS-CoV-2 i) or chronic fatigue syndrome.

### Cardiovascular and Cerebrovascular Diseases

According to one embodiment of the invention, there is provided a formulation of the invention for use in the treatment of cardiovascular and cerebrovascular diseases. For example, cardiovascular and cerebrovascular diseases selected from transient ischemia, hypertension, anti-platelet aggregation, formation of the internal carotid artery onset plate, Cerebral atherosclerosis, arterial atherosclerosis, infarction, myocarditis, meningitis, cerebral arteriosclerosis, hyperlipidaemia and basilar artery insufficiency. In a further embodiment, the cardiovascular and cerebrovascular diseases are selected from cerebral ischemia, cerebral thrombosis, cerebral embolism, cerebral stroke, hypertension and coronary heart disease, myocardial ischemia, arrhythmia, heart failure and angina pectoris.

### Metabolic Diseases

According to one embodiment of the invention, there is provided a formulation of the invention for use in the treatment of metabolic diseases. For example, wherein the metabolic diseases are selected from: diabetes mellitus, obesity, metabolic syndrome, reduced metabolic rate, fatty liver disease (for example NAFLD and NASH), and decline in liver function. In a further embodiment formulation of the invention are useful for weight management.

### Neurological Disorders

According to one embodiment of the invention, there is provided a formulation of the invention for use in the treatment of neurological disorders, including neurodegenerative disorders. Examples of neurological disorders include: cognitive decline, memory decline, anxiety, depression, epilepsy, stroke, amyotrophic lateral sclerosis, dementia (e.g. vascular dementia and Alzheimer's disease), Parkinson's disease. and Huntington disease.

According to a further embodiment of the invention, there is provided a formulation of the invention, for use in the prophylaxis or treatment of a disease state initiated or characterized (i) by a decline in cognitive function; or (ii) by mood disturbances. Such disease states can include, without limitation, neurodegenerative disease, cognitive disorder, mood disorder and stress and/or anxiety disorder.

According to a further embodiment, there is provided a formulation of the invention, for use protecting against brain oxidative stress and/or reducing brain inflammation

### Cancer

According to one embodiment of the invention, there is provided a formulation of the invention for use in the treatment of cancer. According to a further embodiment of the invention, there is provided a formulation of the invention for inhibiting metastasis.

Examples of suitable cancers include: bladder cancer, B-cell lymphoma such as Hodgkin's lymphoma, T-cell lymphoma, T-cell acute lymphoblastic leukaemia, acute lymphoblastic leukaemia, chrome lymphoblastic leukaemia, acute myelogenous leukaemia, chronic myelogenous leukaemia, erythroleukemia, triple negative breast cancer, breast cancer, ovarian cancer, melanoma including paediatric melanoma, lung cancer such as squamous cell lung carcinoma and non small-cell lung cancer, pancreatic cancer, glioblastoma, colorectal cancer, head and neck cancer such a head and neck squamous cell carcinoma, cervical cancer, prostate cancer, liver cancer, oral squamous cell carcinoma, skin cancer, medulloblastoma, hepatocellular carcinoma, intrahepatic and extrahepatic cholangiocarcinoma, desmoid tumours, soft tissue sarcoma, adenoid cystic carcinoma, urothelial cancer, renal cancer, hepatocellular cancer, skin cancer, such as Merkel cell carcinoma, gastric cancer and gastroesophageal cancer.

### Muscle Pathological Conditions

Formulations of the invention find use in the treatment of muscle-related pathological conditions. Therefore, according to a further embodiment of the invention, there is provided a formulation of the invention for use in the treatment or prevention of muscle pathological conditions. Muscle-related conditions include both conditions impacting generally healthy individuals as well as pathological conditions. Such muscle conditions found in healthy people or people affected by a disease include musculoskeletal diseases or disorders; cachexia; muscle wasting; age related decline in muscle function; pre-frailty; frailty; myopathies; neuromuscular diseases, such as Duchenne muscular dystrophy and other dystrophies; age-related sarcopenia; acute sarcopenia; muscle atrophy and/or cachexia, for example muscle atrophy and/or cachexia associated with burns, bed rest, limb immobilization, or major surgery, including thoracic, abdominal, and/or orthopedic surgery; and muscle degenerative disease.

Examples of age-related conditions that may be treated with formulations of the invention include sarcopenia and muscle wasting.

The myopathy may also be caused by a muscular dystrophy syndrome, such as Duchenne.

### Aging/Healthspan

According to a further embodiment of the invention, there is provided a formulation of the invention for use in the increasing or prolonging of healthspan. Healthspan can be defined as the part of a person's life during which they are generally in good health. In a further embodiment of the invention, there is provided a formulation of the invention, for slowing aging.

### Immune Health

In a further embodiment of the invention there is provided the use of a formulation of the invention, for supporting and/or enhancing immune function.

According to a further aspect of the invention there is provided a formulation of the invention, for use in a method of (i) raising an immune response to an antigen and/or (ii) enhancing, modulating or augmenting an immune response to an antigen in a human or animal subject.

According to a further aspect of the invention there is provided a formulation of the invention, for use in a method of preventing, reducing or slowing inflammageing in a human or animal subject.

In a further embodiment of the invention there is provided the use of a formulation of the invention, for use as an anti-inflammatory agent.

According to a further aspect of the invention there is provided a formulation of the invention, for use in a method of treating immunosenescence.

According to a further aspect of the invention there is provided a formulation of the invention, for use in a method of preventing, reducing or slowing stem cell senescence in a human or animal subject.

In a further embodiment of the invention there is provided the use of a formulation of the invention, for supporting and/or enhancing immune function during or after cancer treatment.

In a further embodiment of the invention there is provided the use of a formulation of the invention, for supporting and/or enhancing immune function remission after cancer treatment.

In a further embodiment of the invention there is provided the use of a formulation of the invention, or supporting and/or enhancing immune function during and after hospital admissions.

### Skin

A formulation of the invention for use in the treatment of a skin condition, disease or disorder. Examples of skin diseases, disorders or conditions are selected from the group consisting of melasma, chloasma, hyperpigmentation, skin-aging, liver spots, lentigo, inflammation of the skin, skin irritation, skin infection, warts, psoriasis, and protection of skin from damage caused by the environment and/or therapy. The skin disease, disorder or condition is also selected from melanosis, dermatitis, linea nigra and endocrine diseases such as Addison's and Cushing's syndrome.

In a further embodiment of the invention, skin diseases, disorders and conditions include:
(a) skin aging,
(b) age spots;
(c) liver spots;
(d) dry skin;
(e) radiation induced skin damage (for example, IR, UV, alpha particles, beta particles of gamma irradiation);
(f) lentigo;
(g) hyperpigmentation, for example age-related hyperpigmentation of the skin, or post-inflammatory hyperpigmentation;
(h) melasma (chloasma/mask of pregnancy);
(i) skin irritation, for example, dermatitis;
(j) skin infection, for example, warts;
(k) inflammatory skin conditions (for example, atopic eczema, seborrhoeic eczema, polymorphous photodermatosis, psoriasis, vitiligo),
(l), uneven skin colour (smoothing out thereof),
(m) fine lines and/or wrinkles;
(n) linea nigra;
(o) melanosis;
(p) endocrine diseases, such as Addison's and Cushing's syndrome.

### Hair

In further embodiments of the invention, there is provided a formulation of the invention, for use for treating or preventing hair diseases disorders or conditions.

In one embodiment, hair diseases, disorders and conditions are selected from denutrition-based alopecia, endocrine disorder-based alopecia, vascular disorder-based alopecia, alopecia premature, traction alopecia, alopecia areata, alopecia neurotica, pityriasis alopecia, Trichotillomania, alopecia maligna, female pattern alopecia, male pattern alopecia, androgenetic alopecia, telogen effluvium, tinea capitis, alopecia totalis hypotrichosis, genetic hypotrichosis simplex, systemic drug for alopecia-based hair-loss, mechanical hair-loss, traumatic alopecia, pressure alopecia, anagen effluvium, pityriasis alopecia, alopecia syphilltica, lopecia seborrheica, symptomatic alopecia, alopecia cicatrisata, and alopecia congenita. However, the hair-loss should be understood as meaning including all symptoms classified as the alopecia in this field, regardless of the direct or indirect cause of the occurrence of the hair-loss.

According to a further aspect of the invention there is provided a formulation of the invention, for the treatment of hair loss, wherein the hair loss is caused by one or more of the following: skin disorders, a medicine, a disease, autoimmunity, iron deficiency, severe stress, scalp radiation, pregnancy or pulling at your own hair.

### Treatments:

The formulation of the invention can be taken as a single treatment or, more commonly, as a series of treatments. In one example, a subject takes a dose before or after exercise. For a subject who is not able to exercise, a dose of the formulation may, for example, be taken once, twice or three times per day, or one, two, three, four, five or six times per week. In another example, the intervention may be taken by a subject independent of the subject's ability or need to exercise. It will also be appreciated that the effective dosage of the compound may increase or decrease over the course of a particular treatment.

### Non-Therapeutic Uses

### Muscle Function, Performance, Endurance

The formulation finds use in the management normal physiological function in healthy individuals of conditions characterised by poor physical performance, impaired endurance capacity, and impaired muscle function. Formulations of the invention may improve physical performance in individuals with a disease, including young and elderly individuals. Formulations of the invention may improve physical performance, for example, short-term performance or long-term performance in healthy individuals, including athletes, non-athletic individuals, sedentary individuals and the elderly. This improvement of performance may be measured by the time spent to walk or run a certain distance (for example, an improved performance during the 6 minute walk test (MWT)), an improved time to run a certain distance, an improved IPAQ score on the international physical activity questionnaire, an increased number of chair-stands in a certain time, or another test designed to measure physical performance.

Formulations of the invention further provide for the improvement of endurance capacity. The endurance capacity refers to the time to fatigue when exercising at a constant workload, generally at an intensity<80% VO₂max. Formulations of the invention may improve endurance capacity in individuals with a disease, including young and elderly individuals. Formulations of the invention may improve endurance capacity in healthy individuals, including athletes, non-athletic individuals, sedentary individuals and the elderly. The invention provides for a method of increasing the time to fatigue while performing a specific activity, for example, fitness training, walking, running, swimming, or cycling. This improvement of endurance capacity may be assessed with objective measurements (for example, speed, oxygen consumption or heart rate) or it can be self-reported measurements (for example, using a validated questionnaire).

The invention further provides a formulation to improve, maintain or reduce the loss of muscle function. Formulations of the invention may improve, maintain or reduce the loss of muscle function in individuals with a disease, including young and elderly individuals. Formulations of the invention may improve, maintain or reduce the loss of muscle function in healthy individuals, including athletes, non-athletic individuals, sedentary individuals and the elderly. For example, formulations of the invention may increase muscle strength as evidenced by the improvement of performing a physical activity, such as an exercise, for example, increased ability to lift weights or increased hand grip strength. Also, formulations of the invention may improve muscle structure, for example by increasing or maintaining muscle mass in conditions of normal muscle function, declining muscle function or impaired muscle function.

This invention further provides a formulation to improve the physical performance or endurance capacity as perceived by the individual. For example, by the reduction of in perceived exertion or effort during exercise or an activity as determined using a self-reported questionnaire.

### Muscle Performance

The formulation of the invention is useful in enhancing muscle performance. The invention thus provides a formulation of the invention for use in enhancing muscle performance. The invention also provides a method of enhancing muscle performance by administering to a subject an effective amount of a formulation of the invention. Administration can be self-administration. The enhanced muscle performance may be one or more improved muscle function, improved muscle strength, improved muscle endurance and improved muscle recovery.

The formulation of the invention can thus be used in a method of improving physical endurance (e.g., ability to perform a physical task such as exercise, physical labor, sports activities), inhibiting or retarding physical fatigue, enhancing working capacity and endurance, reducing muscle fatigue, enhancing cardiac and cardiovascular function.

Improved muscle function can be particularly beneficial in elderly subjects with reduced muscle function as a result of an age-related condition. For example, a subject who may benefit from improved muscle function may experience a decline in muscle function which then leads to pre-frailty and frailty. Such subjects may not necessarily experience muscle wastage in addition to their decline in muscle function. Some subjects do experience both muscle wasting and a decline in muscle function, for example subjects with sarcopenia. The formulation of the invention may be used in enhancing muscle performance by administering a formulation of the invention to a subject who is frail or pre-frail.

Muscle performance may be sports performance, which is to say the ability of an athlete's muscles to perform when participating in sports activities. Therefore, in a further embodiment, there is provided a formulation of the invention for enhancing sports performance. Enhanced sports performance, strength, speed, and endurance are measured by an increase in muscular contraction strength, increase in amplitude of muscle contraction, or shortening of muscle reaction time between stimulation and contraction. Athlete refers to an individual who participates in sports at any level and who seeks to achieve an improved level of strength, speed, or endurance in their performance, such as, for example, body builders, bicyclists, long distance runners, and short distance runners. Enhanced sports performance is manifested by the ability to overcome muscle fatigue, ability to maintain activity for longer periods of time, and have a more effective workout.

In a further embodiment, there is provided a formulation of the invention for use in a method of enhancing physical performance in a subject, for example, in an elite athlete or sub-elite athlete.

According to a further embodiment of the invention, there is provided a formulation of the invention, for use in a method of enhancing physical performance in a subject, for example, in an elite athlete or sub-elite athlete.

In certain embodiments, enhancing physical performance comprises at least one effect selected from the group consisting of enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage. In further embodiments, enhancing physical performance comprises enhancing muscle performance during a high-intensity aerobic activity. In yet further embodiments, enhancing physical performance comprises increasing aerobic endurance during a high-intensity aerobic activity. In still further embodiments, enhancing physical performance comprises increasing resting metabolic rate (RMR). In certain embodiments, enhancing physical performance results in an improvement in athletic performance. In further embodiments, enhancing physical performance results in an improvement in footrace completion times. In yet further embodiments, enhancing physical performance results in a decrease in Ratings of Perceived Exertion (RPE).

According to a further embodiment of the invention, there is provided a formulation of the invention for use in a method of enhancing physical recovery in a subject, for example, in an elite athlete or sub-elite athlete.

In certain embodiments, physical recovery is enhanced after a high-intensity aerobic activity. In further embodiments, enhancing physical recovery comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage. In yet further embodiments, enhancing physical recovery comprises enhancing muscle recovery after a high-intensity aerobic activity. In still further embodiments, enhancing physical recovery comprises reducing muscle soreness after a high-intensity aerobic activity. In certain embodiments, enhancing physical recovery comprises lowering creatine kinase (CK) levels in the subject, compared to baseline, following an aerobic activity as measured by area under the plasma concentration-time curve of CK (AUCCK). In further embodiments, enhancing recovery comprises lowering C-reactive protein (CRP) levels in the subject, compared to baseline, following an aerobic activity as measured by area under the plasma concentration-time curve (AUCCRP).

### Immune Function

In a further embodiment of the invention there is provided the use of a formulation of the invention, for maintaining or enhancing immune health.

In a further embodiment of the invention there is provided the use of a formulation of the invention, for reducing or slowing inflammaging.

In a further embodiment of the invention there is provided the use of a formulation of the invention, for slowing immune aging.

In a further embodiment of the invention there is provided the use of a formulation of the invention, for reducing immune cell aging.

In a further embodiment of the invention there is provided the use of a formulation of the invention, for boosting immune function.

In a further embodiment of the invention there is provided the use of a formulation of the invention, for supporting a healthy immune system.

In a further embodiment of the invention there is provided the use of a formulation of the invention, for prevention or management of colds and/or flu.

In a further embodiment of the invention there is provided the use of a formulation of the invention, for the treatment of infection.

In a further embodiment of the invention there is provided the use of a formulation of the invention, for improving vaccination response.

In a further embodiment of the invention there is provided the use of a formulation of the invention, for enhancing an immune response against infection.

In a further embodiment of the invention there is provided the use of a formulation of the invention, for protecting against infection, for example, protection against colds and flu.

In a further embodiment of the invention there is provided the use of a formulation of the invention, for immune support.

In a further embodiment of the invention there is provided the use of a formulation of the invention, for reducing the risk of respiratory tract infections (for example, colds, flu or pneumonia).

In a further embodiment of the invention there is provided the use of a formulation of the invention, for increasing the rate of recovery from respiratory tract infections (for example, colds, flu or pneumonia).

In a further embodiment of the invention there is provided the use of a formulation of the invention, for increasing CD8 positive T-cells, for example, naïve CD8-positive T-cells.

Brain function According to a further embodiment of the invention there is provided the use of a formulation of the invention, for maintaining or enhancing brain health.

According to a further embodiment of the invention there is provided the use of a formulation of the invention, for promoting healthy brain function.

According to a further embodiment, there is provided a formulation of the invention, for use for optimising brain health.

According to a further embodiment, there is provided a formulation of the invention, for use to improve, protect, and maintain brain function and cognition.

According to a further embodiment, there is provided a formulation of the invention, for use for enhancing mental alertness, for example, mental accuracy and clearer/sharper thinking.

According to a further embodiment, there is provided a formulation of the invention, for use for increasing psychomotor speed and/or reaction time.

According to a further embodiment, there is provided a formulation of the invention, for use for enhancing/improving memory.

According to a further embodiment, there is provided a formulation of the invention, for use for enhancing/improving learning.

According to a further embodiment, there is provided a formulation of the invention, for use for enhancing/improving reasoning, for example, promoting mental processing.

According to a further embodiment, there is provided a formulation of the invention, for use for enhancing/improving focus, for example, concentration, complex attention and/or sustained attention.

According to a further embodiment, there is provided the use of a formulation of the invention, for stress relief and/or anxiety relief.

According to a further embodiment, there is provided the use of a formulation of the invention, for treating stress and/or anxiety.

According to a further embodiment, there is provided the use of a formulation of the invention, for treating depression.

According to a further embodiment, there is provided the use of a formulation of the invention, for improving mood.

According to a further embodiment, there is provided the use of a formulation of the invention, for enhancing sleep and/or relaxation.

According to a further embodiment, there is provided the use of a formulation of the invention, for improving cognitive function. In one embodiment, the cognitive function is selected from the group consisting of perception, memory, attention, speech comprehension, speech generation, reading comprehension, creation of imagery, learning, and reasoning. In one embodiment, the cognitive function is selected from the group consisting of perception, memory, attention, and reasoning. In one embodiment, the cognitive function is memory.

According to a further embodiment, there is provided a formulation of the invention, for use to improve, protect, and maintain brain function and cognition.

### Skin

According to a further embodiment, there is provided the use of a formulation of the invention, for treating or preventing skin conditions in generally healthy subjects. For example, it is suitable for use by subjects with skin prone to the ailments mentioned herein, for example subjects with intermittent skin issues, for example, skin prone to sunburn, prone to acne and prone to inflammation.

According to a further embodiment of the invention, there is provided use of a formulation of the invention, for one or more of the following:
a) maintaining or enhancing skin health and appearance;
b) for maintaining or enhancing skin energy;
c) for maintaining or enhancing skin collagen;
d) for maintaining or enhancing skin elasticity
e) reducing skin biological aging;
f) supporting healthy skin aging;
g) reducing skin wrinkles, and/or fine lines;
h) improving skin mitochondrial function;
i) maintaining or improving skin hydration;
j) promoting replenishment of skin ceramide levels;
k) improving barrier function;
l) protecting against free radical damage; and/or
m) improving skin dryness;
n) improving photodamaged skin;
o) Improving skin health in skin prone to redness, inflammation, acne or dryness
p) helping replenish ceramide stores; and
q) reducing skin redness and irritation.

In a further embodiment of the invention, there is provided a formulation of the invention, for one or more of the following uses:
(a) skin bleaching and/or lightening skin colour and/or lightening skin tone;
(b) skin whitening;
(c) protection of skin caused by damage by the environment (For example, damage caused by sunlight/UV irradiation and/or damage caused by pollution);
(d) decreasing pigmentation; and
(e) suppressing melanin production.

In a further embodiment of the invention, there is provided a formulation of the invention, for one or more of the following uses:
(a) renewal and/or revitalization of skin appearance;
(b) enhancement of skin energy supply;
(c) improvement in skin texture;
(d) enhancement of skin radiance;
(e) skin rejuvenation/regeneration;
(f) reduction of pore and micro line visibility;
(g) improvement in skin respiration.
(h) support optimal cellular function in skin;
(i) promote/activate skin detoxifying processes;
(j) promote collagen repair, and/or
(k) improve skin stem cell health.

### Hair

According to a further aspect of the invention there is provided a formulation of the invention, for stimulating hair growth.

According to a further aspect of the invention there is provided a formulation of the invention for the treatment of hair loss

According to a further aspect of the invention there is provided a formulation of the invention, for preventing or ameliorating hair loss.

According to a further aspect of the invention there is a formulation of the invention, for delaying the onset of hair loss.

According to a further aspect of the invention there is provided a formulation of the invention, for treating hair thinning.

According to a further aspect of the invention there is provided a formulation of the invention, for use in a method of increasing hair thickness (i.e. number of hair fibres per surface area) and/or number hair follicles actively producing hair fibres.

According to a further aspect of the invention there is provided a formulation of the invention, for slowing or preventing premature greying of hair.

In a further embodiment of the invention, there is provided a formulation of the invention, for one or more of the following uses:
(a) maintaining or enhancing hair thickness;
(b) hair follicle cell regeneration;
(c) hair follicle cell survival;
(d) hair stem cell growth and/or regeneration;
(e) hair cell survival;
(f) hair loss prevention,
(g) promoting new hair growth;
(h) hair maintenance;
(i) scalp health improvement,
(j) improving or maintaining hair strength,
(k) or improving survival of hair transplants; and/or
(l) hair growth restoration promotion.

In a further embodiment of the invention, there is provided a formulation of the invention, for one or more of the following uses:

a) enhancing hair stem cell function;
b) enhancing hair follicle elongation,
c) enhancing hair matrix proliferation, for example, as measured by Ki67 levels;
d) enhancing hair growing phase, (enhancing the duration of the anaphase);
e) hair matrix proliferation,
f) inhibition of hair matrix apoptosis; and
g) reducing melanin clumping, for example, in pigmentary units, for example, in hair follicle pigmentary units.

### Nails

Formulations of the invention find use in the treatment or prevention of diseases, disorders and conditions associated with nails. Such uses in diseases, disorders and conditions include use in both pathological and non-pathological conditions and cosmetic indications.

In a further embodiment of the invention, nail diseases, disorders and conditions include nail deformities (changes in nail shape) and nail dystrophies (changes in nail texture, colour or both.

Nail conditions include: brittle nails, triangular worn down nails, trachyonychia, and habit tic deformity.

Therefore, according to a further embodiment of the invention, there is provided a formulation of the invention, for use in the treatment or prevention of diseases, disorders and conditions associated with nails, for example, nail deformities (changes in nail shape) and nail dystrophies (changes in nail texture, colour or both and brittle nails, triangular worn down nails, trachyonychia, and habit tic deformity.

### Urolithin Administration/Dosage Regimes

The combinations of the present disclosure involve oral administration of a urolithin of formula (I) or salt thereof to a subject in a daily amount in the range of 1.7 to 6.0 mmol per day, for example, from 1.7 to 2.7 mmol per day, or from 2.8 to 6.0 mmol per day, for a period between 2 to 16 weeks prior to vaccination. As discussed below, administration of is preferred in the range 250 mg to 1000 mg urolithin A (which corresponds to about 1.1 to 4.4 mmol) results in a surprisingly good pharmacokinetic profile, compared with a much higher dosage of 2000 mg. In one embodiment the dose is 250 mg/day, in an alternative embodiment the dose is 500 mg/day and in another embodiment the dose is 1000 mg/day.

In a further embodiment, administration doses are selected from:
250 mg once or twice a day;
500 mg once or twice a day;
750 mg once or twice a day;
1000 mg once or twice a day;
1250 mg once or twice a day; or
1500 mg once or twice a day

The methods of the present disclosure involve daily administration of the compound of formula (I) or salt thereof, or of a formulation containing the compound or salt. In some embodiments the compound or formulation is administered once per day, i.e. the compound or formulation is to be administered at least once per 24 hour period. In other embodiments the compound, or formulation comprising the compound, is administered multiple times per day, for example twice per day, or three or four times per day. In such cases, the daily dosage is divided between those multiple doses. In one embodiment administration is once a day, in a second embodiment administration is twice a day, in a third embodiment administration is three times a day.

The methods of the present disclosure would usually require daily administration of the compound of formula (I) or salt thereof, or of a formulation containing the compound or salt, for a period over several months. In some embodiments, the methods may involve administration of the compound of formula (I), or salt thereof, over for example daily for at least 2 weeks, 3 weeks, 4 weeks, 5 weeks, 6 weeks, 8 weeks, 12 weeks, 4 months, 6 months, or for at least a year. In some embodiments, the method comprises administering the compound or salt thereof daily for a period of up to 3 months, up to 6 months, up to 1 year, up to 2 years or up to 5 years. In some embodiments, the method comprises administering the compound or salt daily for a period in the range of from 21 days to 5 years, from 21 days to 2 years, from 21 days to 1 year, from 21 days to 6 months, from 21 days to 12 weeks, from 28 days to 5 years, from 28 days to 2 years, from 28 days to 1 year, from 28 days to 6 months, from 28 days to 4 months, from 28 days to 12 weeks, 6 weeks to 2 years, from 6 weeks to 1 year, from 8 weeks to 1 year, or from 8 weeks to 6 months.

The methods of the present disclosure require daily administration of an amount of compound of formula (I) or salt thereof, of from 0.7 mmol per day up to 2.7 mmol per day thereof or from 0.7 mmol twice per day up to 2.7 mmol twice a day. In some embodiments, the amount administered is in the range of from 2.0 to 2.5 mmol. In some embodiments, the amount administered is approximately, 1.1, 1.2, 1.3, 1.4. 1.5, 1.6 1.7, 1.8, 1.9, 2.0, 2.1, 2.2, 2.3, 2.4, 2.5, 2.6, or 2.7 mmol per day. In other embodiments, the amount administered is approximately, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 mmol per day. In some preferred embodiments the method involves administration of approximately 2.2 mmol per day or 2.2 mmol twice per day of the compound of formula (I) or salt thereof (e.g. of urolithin A). The exact weight of compound that is administered depends on the molecular weight of the compound that is used. For example, urolithin A has a molecular weight of 228 g/mol (such that 2.20 mmol is 501.6 mg) and urolithin B has a molecular weight of 212 g/mol (such that 2.20 mmol is 466.4 mg).

In a further embodiment, the methods of the present disclosure require daily administration of an amount of compound of formula (I) or salt thereof, of from 2.8 mmol per day up to 6.0 mmol per day or twice per day thereof. In some embodiments, the amount administered is in the range of from 4.0 to 4.8 mmol. In some embodiments, the amount administered is approximately, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 mmol. In some preferred embodiments the method involves administration of approximately 4.4 mmol per day or twice per day of the compound of formula (I) or salt thereof (e.g. of urolithin A). The exact weight of compound that is administered depends on the molecular weight of the compound that is used. For example, urolithin A has a molecular weight of 228 g/mol (such that 4.40 mmol is 1003.2 mg) and urolithin B has a molecular weight of 212 g/mol (such that 4.40 mmol is 932.8 mg).

In some embodiments the methods involve administration of urolithin A in an amount in the range of from 400 to 600 mg/day or 400 to 600 mg once per day. In a preferred embodiment the method involves administration of urolithin A in an amount in the range of from 450 to 550 mg, more preferably approximately 500 mg per day.

In other embodiments the methods involve administration of urolithin A in an amount in the range of from 700 to 1300 mg/day per day, or in the range of from 750 to 1250 mg, or in the range of from 800 to 1200 mg, or in the range of from 850 to 1150 mg, or in the range of from 900 to 1100 mg per day. In a preferred embodiment the method involves administration of urolithin A in an amount in the range of from 950 to 1150 mg/day, more preferably approximately 1000 mg/day.

In some preferred embodiments, the methods involve administering urolithin A to the subject in an amount in the range of from 4.5 to 11 mg/kg/day, such as 4.5 to 8.5 mg/kg/day. In another embodiment, the methods involve administering urolithin A to the subject in an amount in the range of 5 to 9 mg/kg/day. In another embodiment, the methods involve administering urolithin A to the subject in an amount in the range of from 6.0 to 8 mg/kg/day.

In other preferred embodiments, the methods involve administering urolithin A to the subject in an amount in the range of from 9 to 18 mg/kg/day such as 9 to 17 mg/kg/day. In another embodiment, the methods involve administering urolithin A to the subject in an amount in the range of from 10 to 17 mg/kg/day. In another embodiment, the methods involve administering urolithin A to the subject in an amount in the range of from 11 to 18 mg/kg/day.

Dosage regimes which combine a 500 mg dose and a 1000 mg dose may be advantageous. For example, a twice daily dosage regime which combines a first dose of 1000 mg and a second dose several hours later of 500 mg. Said 500 mg dose may be 6-18 hours after the 1000 mg dose, for example 8-12 hours after the 1000 mg dose. For example, about 12 hours after the 1000 mg dose. Thus, according to a further aspect of the invention there is provided the treatment of a disease with a compound of Formula (I) which comprises a twice daily dosage regime comprising a first dose of 1000 mg, followed by a second dose of 500 mg wherein the two doses are separated by 6-18 hours.

The compound of formula (I) or salt thereof, or formulation containing the compound of salt, may be administered at any suitable time, for example it may be administered in the morning after sleep or in the evening. In some embodiments it may be preferable for the method to be performed at approximately the same time(s) each day, for example within 15, 30, 60 or 120 minutes of a given time point.

The appropriate dose of a formulation of the invention is chosen based on clinical indications by a treating physician or the non-therapeutic treatment.

In some preferred embodiments, formulations of the invention comprises a compound of formula (I) or salt thereof (e.g. urolithin A), with a preferred particle size distribution. A particular particle size distribution enables the compound of formula (I) to disperse or dissolve more rapidly. A particular particle size distribution can be achieved by methods established in the art, for example compressive force milling, hamermilling, universal or pin milling, or jet milling (for example spiral jet milling or fluidised-bed jet milling) may be used. Jet milling is especially suitable. Furthermore, a particular particle size distribution may also be directly derived by the use of a particular chemical process. If a particular particle size distribution is used, then preferably the compound has a D₅₀ size of under 100 μm—that is to say that 50% of the compound by mass has a particle diameter size of under 100 μm. More preferably, the compound has a D₅₀ size of under 75 μm, for example under 50 μm, for example under 25 μm, for example under 20 μm, for example under 10 μm. More preferably, the compound has a D₅₀ in the range 0.5-50 μm, for example 0.5 to 20 μm, for example 0.5 to 10 μm, for example 1.0 to 10 μm, for example 1.5 to 7.5 μm, for example 2.8 to 5.5 μm. Preferably, the compound has a D₅₀ size of under 100 μm. More preferably, the compound has a D₅₀ size of under 75 μm, for example under 50 μm, for example under 25 μm, for example under 20 μm, for example under 15 μm. The compound preferably has a D₉₀ in the range 5 to 100 μm, for example 5 to 50 μm, for example 5 to 20 μm, for example 7.5 to 15 μm, for example 8.2 to 16.0 μm. Preferably, the compound has a D₁₀ in the range 0.5-1.0 μm. Preferably, the compound of formula (I) or salt thereof (e.g. urolithin A) has a D₉₀ in the range 8.2 to 16.0 μm, a D₅₀ in the range 2.8 to 5.5 μm and a D₁₀ in the range 0.5 to 1.0 μm.

In a further embodiment, the compound of formula (I) or salt thereof has a particle size distribution selected from one of the following:
(i) D₅₀ size in the range 0.5 to 50 μm and a D₉₀ size in the range 5 to 100 μm,
(ii) the compound has a D₉₀ size in the range 8.2 to 16.0 μm, a D₅₀ size in the range 2.8 to 5.5 μm and a D₁₀ size in the range 0.5 to 1.0 μm;
(iii) the compound of Formula (I) has a D₅₀ size in the range 0.5 to 20 μm and a D₉₀ size in the range 5 to 50 μm;
(iv) the compound of Formula (I) has a D₅₀ size under 50 μm and a D₉₀ size under 75 μm;
(v) the compound of Formula (I) has a D₅₀ size under 25 μm and a D₉₀ size under 50 μm;
(iv) the compound of Formula (I) has a D₅₀ size under 10 μm and a D₉₀ size under 20 μm;
(v) the compound of Formula (I) has a D₅₀ size under 10 μm and a D₉₀ size under 15 μm; or
(vi) the compound of Formula (I) has a D₅₀ size of 10 μm and a D₉₀ size of 20 μm.

Pharmaceutical formulations containing the compound of formula (I) or salt thereof may for example include additional pharmaceutically active compounds.

A unit dose formulation used in the methods described herein preferably contains 250 mg or 500 mg of the compound of formula (I), for example 250 mg or 500 mg of urolithin A.

The term ‘about’ refers to a tolerance of ±20% of the relevant value, for example±15% of the relevant value, such as ±10% of the relevant value or ±5% of the relevant value.

The term ‘alginate’ refers to salts of alginic acid, for example, sodium or calcium salts.

The term ‘artificial sweetener’ refers to any sweetener, which does not occur in nature The term “carrier” refers to a diluent, adjuvant, excipient, or vehicle with which the therapeutic is administered.

The terms ‘composition’ and ‘formulation’ are used interchangeably herein.

The term ‘fibre’ refers to plant derived carbohydrates which the human digestive system is incapable of breaking down.

The term ‘esterification range’ (DE—degree of esterification) when related to pectin refers to the percentage of esterified carboxyl groups to total carboxyl groups in pectin. Pectin is divided into low-ester pectin (DE<50%) and high-ester pectin (DE>50%)

The term ‘excipient’ refers to a substance formulated alongside the active ingredient of a medication, included, for example, for the purpose of long-term stabilization, bulking up solid formulations that contain potent active ingredients in small amounts (thus often referred to as “bulking agents”, “fillers”, or “diluents”), or to confer a therapeutic enhancement on the active ingredient in the final dosage form, such as facilitating drug absorption, reducing viscosity or enhancing solubility.

The term ‘Frutalose®’ refers to a powdered oligofructose produced by partial hydrolysis of chicory inulin. When used herein as well as referring to the specific product, Frutalose®, the term ‘Frutalose®’ also refers to any oligofructose prepared by partial hydrolysis (for example, enzymatic hydrolysis) of a larger polymer, such as inulin, for example by partial hydrolysis of inulin.

The term ‘gelling component’ refers to one or more components which serves to form the polymeric matrix providing the chewable texture to the formulation.

The term ‘Gum Arabic’ or ‘Arabic Gum’ refers to a tree gum exuded by species of Acacia.

In one embodiment the exudate is from the trunks and/or branches of Acacia senegal and/or Vachellia (Acacia) seyal.

In a further embodiment, the Gum Arabic is selected from the dried exudate from the trunks and/or branches of Acacia senegal and/or Vachellia (Acacia) seyal in the family Fabaceae (Leguminosae as defined by the 31st Codex Committee for Food Additives, held at The Hague from 19 to 23 Mar. 1999. In a further embodiment, the term ‘Gum Arabic’ comprises such exudates, whether dried or not dried.

In a further embodiment, the Gum Arabic is selected from the dried exudate from the trunks and/or branches of Acacia Senegal, Vachellia (Acacia) seyal, Acacia polyacantha, Acacia laeta and/or Acacia mellifera. In a further embodiment, the term ‘Gum Arabic’ further comprises the exudate, for example, dried exudates from the trunks and/or branches of Combretum and/or Albizia.

In a further embodiment, the Gum Arabic is from the dried exudate from the trunks and/or branches of Acacia Senegal.

Furthermore, the term ‘Gum Arabic’ comprises plant exudate which comprises a complex mixture of glycoproteins and polysaccharides, predominantly polymers of arabinose and galactose, with utility in the formulation of urolithins, particularly urolithin A, particularly with utility in formulating urolithins for administration in water, whether dissolved, as a suspension or any other chemical form.

In a further embodiment, the term ‘ Gum Arabic’ comprises any synthetic equivalent of a Gum Arabic, as defined herein.

The terms “gummy” refers to a dosage form which retains its integrity and texture upon chewing, does not initially break into discrete, solid particulates upon chewing, utilizes a gelling matrix, and is intended to be swallowed. The term ‘chewable formulation’ refers to a gummy.

The term gummy is distinct from a jelly which is a much softer gelatinous matrix, made with, for example, agar.

The term ‘healthspan’ refers to the number of years that someone lives or can expect to live in reasonably good health.

The term ‘pectin’ refers to a mixture of complex polysaccharides that are present in the primary cell walls of a plant, and are abundant in the green parts of terrestrial plants. Pectin may be high methoxy pectin or low methoxy pectin. The term pectin further comprises amidated pectin, for example, amidated low methoxy pectin.

The term “pharmaceutically acceptable” means approved by a regulatory agency of the Federal or a state government or listed in the U.S. Pharmacopeia or other generally recognized pharmacopeia for use in animals, and more particularly in humans.

The term, “separate” administration means the administration of each of two or more compounds to a patient from non-fixed dose dosage forms simultaneously, substantially concurrently, or sequentially in any order. There may, or may not, be a specified time interval for administration of each the compounds.

The term “sequential” administration means the administration of each of two or more compounds to a patient from non-fixed (separate) dosage forms in separate actions. The administration actions may, or may not, be linked by a specified time interval. For example, administering compounds over a specified time such as once every 14 to 21 days.

The term “simultaneous” administration means the administration of each of two or more compounds to a patient in a single action such as where each compound are administered independently at substantially the same time or separately within time intervals that allow the compounds to show’ a cooperative therapeutic effect.

The term ‘sweetener system’ refers to a sweetener or combination of sweeteners. In this specification the integers ‘formulation’ and ‘composition’ are used interchangeably.

### EXAMPLES

The invention will now be illustrated with respect to the following non-limiting examples

### Example 1: Pectin Formulation

An example formulation of the invention consists of

| | % by total |
|---|---|
| Ingredients | ingredient mass |
| Allulose Crystalline Powder, organic | 33.54% |
| Water (soft, filtered, municipal) | 19.98% |
| Apple Juice Concentrate, 70 Brix | 19.38% |
| Urolithin A | 9.17% |
| Elderberry Juice Concentrate, | 3.73% |
| 65 Brix, organic | |
| Soluble Tapioca Fibre powder, organic | 3.73% |
| Apple Puree, 38 Brix | 3.50% |
| Ascorbic Acid, Fine Granular | 2.42% |
| Pectin, Slow-Set High-Methoxyl | 1.64% |
| Sodium Ascorbate | 1.36% |
| Clarified Lemon Juice Concentrate, 44 Brix | 0.86% |
| Zinc Citrate, Dihydrate Purified | 0.29% |
| Selenium Methionine | 0.09% |
| Vitamin D3 100 CWD EU | 0.07% |
| Flavours | Minimum 0.23% |

### Example 2: Process for the Preparation of the Formulation of Example 1

1) Blend fruit concentrates, purees, water, and sodium ascorbate together in a container larger enough to hold them.
2) Blend pectin and 10 times its weight of allulose together in a container large enough to hold them.
3) Blend the following dry ingredients together in a container large enough to hold them (i.e., the remainder of allulose, the urolithin A, all vitamins (except the ascorbic acid), all minerals, and the tapioca fibre)
4) Measure out lemon concentrate and add the natural flavour to it.
5) Measure out the ascorbic acid.
6) Measure out the colour and blend it into just enough water to create a thick liquid.
7) Add the ingredients from #1 above into the confectionery kettle at room temperature.
8) Gradually whisk the ingredients from #2 above into the ingredients in the kettle. Allow to hydrate for 5-10 minutes and whisk well once more.
9) Gradually whisk the ingredients from #3 above into the ingredients in the kettle.
10) While monitoring the temperature of the gummy solution, heat relatively rapidly while stirring the mixture, avoiding extreme temperatures that could cause scorching.
11) When the solution begins to boil, pay close attention to the temperature, which should eventually rise to 230 F.
12) Immediately remove the gummy solution from the heat at this time.
13) Once cooled to approximately 220 F, stir in the following ingredients until homogenous: lemon concentrate with the natural flavour and the ascorbic acid.
14) Finally, add the liquid colour and stir until homogenous.
15) Transfer the well mixed gummy solution to a heated holding tank with agitator in preparation for depositing into molds. Temperature should be maintained such that the high-methoxyl pectin does not begin to gel prior to molding.
16) Cool the deposited gummy solution in the molds until gelation has occurred and they are firm enough to be demolded and a carnauba wax or other coating applied via panner or other means to keep the gummies from sticking.

### Example 3: Pectin Formulation

An example formulation of the invention comprises:

| Ingredients | g per dose | |
|---|---|---|
| Allulose Crystalline Powder, organic | 1.84 | g |
| Urolithin A | 500 | mg |
| Soluble Tapioca Fibre powder, organic | 0.21 | g |
| Pectin, Slow-Set High-Methoxyl | 0.14 | g |
| Zinc bisglycinate chelate | 6 | mg |
| Selenium Methionine | 30 | μg |
| Vitamin C | 226 | mg |
| Vitamin D3 100 CWD EU | 11 | μg |

| | |
|---|---|
| Flavours | Minimum 0.23% |

The formulation further comprises water, Apple Juice Concentrate, Clarified Lemon Juice Concentrate, Elderberry Juice Concentrate and Apple Puree and flavourings (minimum 0.23% (w/w).

### Example 4: Preparation of Spray Dried Urolithin a Compositions

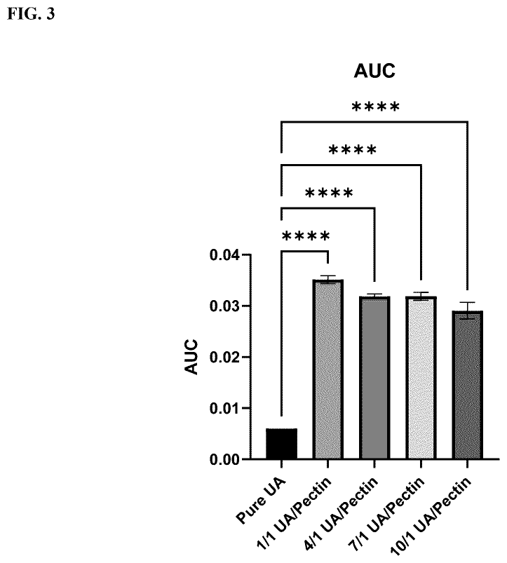

Ethanol was used as solvent to prepare a stable UA suspension and a stable Citrus pectin (lot number SLBV5461, Sigma Life Science) suspension. Both suspensions were stirred minimal for 1 hour. Suspension were mixed to obtain suspension in which UA and Citrus pectin were at a w/w ratio of either UA/pectin 1/3 or 1/1. Following spray drying, the two resulting UA/Pectin powder formulations were analyzed for their solubility when dissolved in a biorelevant medium recapitulating a gut-like environment (FaSSIF, ph=6.5). Solubility of the UA/Pectin formulations were compared to solubility of pure UA powder. For both pure UA powder and UA/pectin formulations, an equivalent dose of 10 mg UA was added to FaSSIF medium. Solubility was analysed by UPLC method (Ultra-Performance Liquid Chromatography) at different time points after addition of powders to the FaSSIF medium. UA/Pectin powders showed a strikingly enhanced solubility profile at both ratios tested, when compared to pure UA powder (FIG. 3).

We further tested UA/Pectin based formulation using water as solvent, instead of ethanol. UA was dissolved in water to form a stable suspension. Citrus pectin (lot number SLBV5461, Sigma Life Science) was dissolved in water to form a solution. The UA suspension in water was complexed with Citrus pectin solution (lot number SLBV5461, Sigma Life Science) at different ratios. One ratio w/w was UA/Pectin 1/1 that showed positive data in the formulation using EtOH as solvent. We also tested a formulation with lower Pectin content, in particular formulations with UA/pectin ratio: 4/1 w/w, 7/1 w/w, 10/1 w/w.

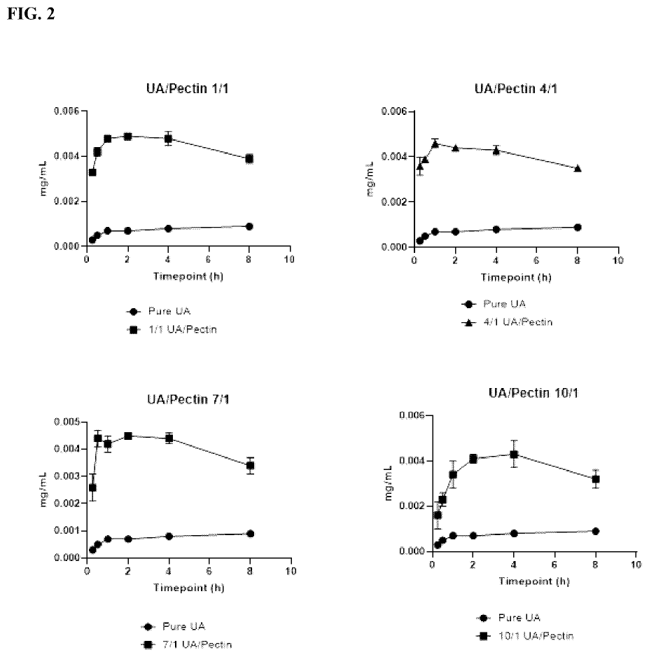

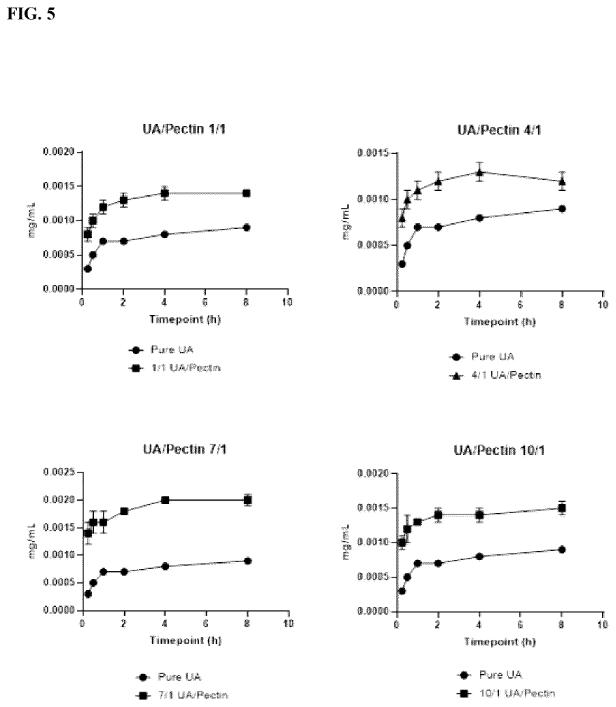

Powders containing the equivalent of 10 mg UA complexed with pectin at different ratio and 10 mg of pure UA powder were analyzed for their solubility profile when dissolved in a biorelevant medium recapitulating a gut-like environment (FaSSIF, ph=6.5). Solubility was analysed by UPLC method Ultra-Performance Liquid Chromatography at different time points after addition of powders to the FaSSIF medium. UA/Pectin powders showed a strikingly enhanced solubility profile at all ratios tested (FIG. 2). This was confirmed by calculating the area under the curve (AUC) of the dissolution profiles, in order to quantify the total presence of soluble UA over time. All UA/Pectin powders showed a significant increase in AUC (FIG. 5). Surprisingly the increase in AUC was not proportional, with ratio 1:1 showing the highest AUC, the 1:4 and 1:7 ratios showing similar profiles and solubility enhancement starting to decrease only when lowering the pectin ratio to UA/Pectin 10/1 (FIG. 5).

To compare the ability to increase UA solubility of the different formulations, we also calculated the maximum concentration of UA in solution achieved comparing formulations with UA prepared in Ethanol or in water (Cmax).

For the formulations using ethanol as solvent, Cmax was 0.0009 for the pure UA powder, 0.0038 for the UA/Pectin 1/3 powder and 0.0035 for the UA/Pectin 1/1 powder. This indicates that complexing UA with pectin—using ethanol as solvent—allows an increase in solubility Cmax by 322% with the ratio UA/pectin 1/3 and by 288% with the ratio UA/pectin 1/1 (FIG. 5).

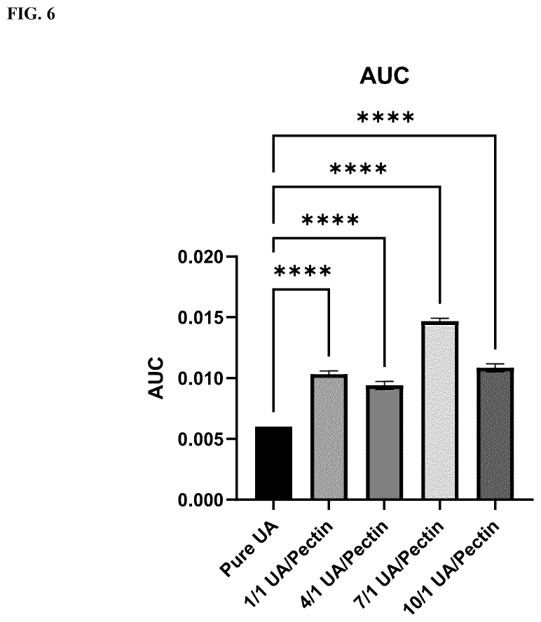

For the formulations using water as solvent, Cmax was 0.0009 for the pure UA powder, 0.0049 for the UA/Pectin 1/1 powder, 0.0046 for the UA/Pectin 4/1 powder, 0.0045 for the UA/Pectin 7/1 powder, 0.0043 for the UA/Pectin 10/1 powder. This indicates that complexing UA with pectin—using ethanol as solvent—allows an increase in solubility Cmax by 444% with the ratio UA/pectin 1/1, by 411% with the ratio UA/pectin 4/1, by 400% with the ratio UA/pectin 7/1, by 377.78% with the ratio UA/pectin 10/1 (FIG. 6).

This data indicates that using water as solvent leads to formulations with better UA solubility compared to using ethanol as solvent, after spray drying.

Formulations tested above have been generated by spray drying solutions/suspensions of UA and Pectin. The rationale was to improve the solubility of UA by reducing its crystalline state in favour of an amorphous state.

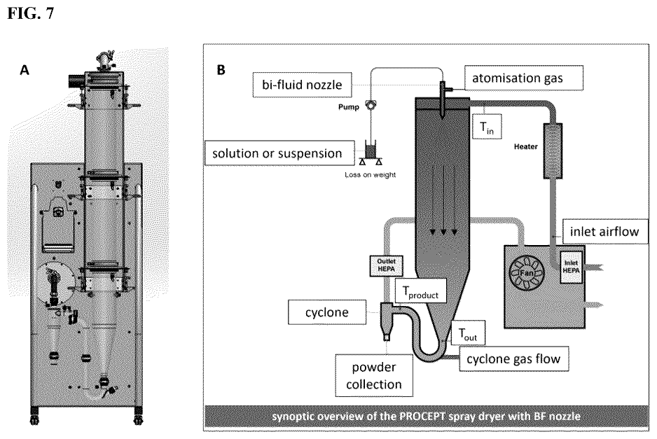

We also determined the solubility of UA following simple physical mixture of UA and Pectin, without spray drying. Surprisingly, all formulations tested showed increased UA solubility despite spray drying not being used (FIG. 7).

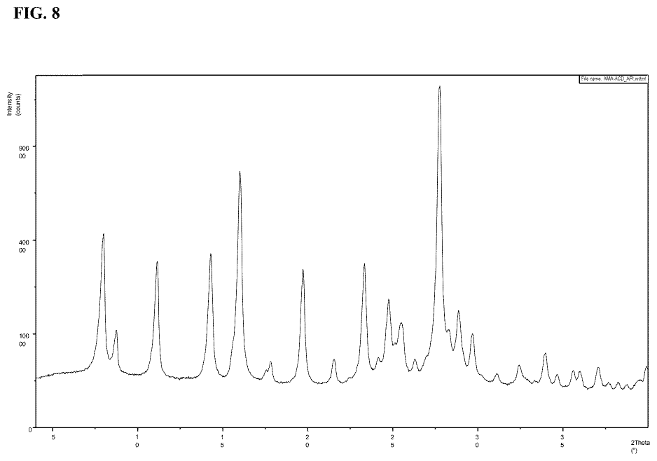

This was confirmed by calculating the area under the curve (AUC) of the dissolution profiles, in order to quantify the total presence of soluble UA over time. All UA/Pectin powders showed a significant increase in AUC (FIG. 6). Surprisingly the increase in AUC was not proportional, with ratio 7:1 showing the highest AUC (FIG. 8).

In summary data indicates that UA solubility can be increased significantly when UA is spray dried at certain ratio with pectin as excipient. Solubility enhancement is more pronounced with water is used as solvent. In addition, we show that even simple physical mixing of UA and pectin at certain ratio allow UA solubility improvement, with UA/Pectin 7/1 being the ratio leading to the best results.

### Example 5: Methodology for Preparation of Spray-Dried Urolithin a Formulations

### 1. Equipment Set-Up

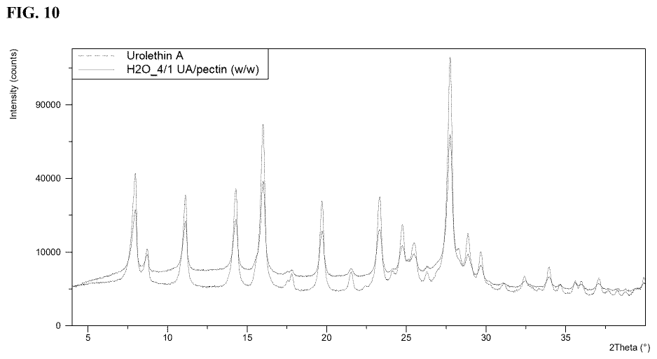

Spray drying trials were performed on the PROCEPT 4M8-TRIX spray dryer open loop system (i.e. atmospheric conditions/compressed gas) (FIG. 10). The spray dryer was equipped with a large cyclone. The extended column was installed to increase the residence time of the droplets/particles in the heated chamber. For all trials a bi-fluid nozzle with a 1.0 mm orifice was used.

### 2. Preparation Solutions and Suspensions

All suspensions were prepared with a solid load of 4.00% (w/w) in a total quantity of 50 g. For each formulation, this quantity was completely spray dried, resulting in 2 g of total dosed solids. Depending on the used UA/pectin w/w ratio, different amounts of UA and pectin were added.

For solvents ethanol and water, UA was firstly suspended in the solvent, followed by addition of the excipient Pectin. If UA and/or the excipient did not dissolve, formulation was spray dried as a suspension. Suspension was magnetically stirred for minimal 1 hour prior to processing. Also, during the spray drying process, suspensions were continuously stirred.

The non spray dried formulations were prepared by addition of UA powder and Pectin powder at different ratio tested. Powders were mixed using mortar and pestle until content was visually uniform.

### Example 6: XRPD Evaluation of Urolithin A Samples

Urolithin A crystallinity was measured using X-ray powder diffraction (XRPD). XRPD diffractograms of crystalline urolithin A show a number of peak indicative of the crystal structure.

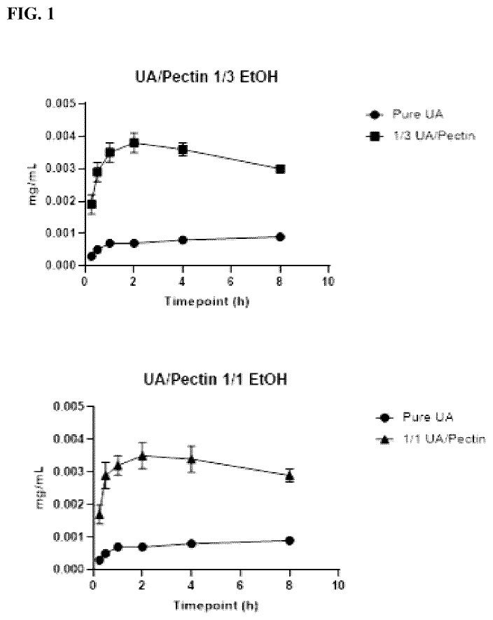

In amorphous material these peaks are lost or at the very least much reduced. For example, an XRPD diffractogram of crystalline urolithin A is shown in FIG. 1.

The XRPD analysis was conducted under the following conditions.
Equipment: Aeris diffractometer (PANalytical, malvern) equipped with Cu tube (Kα λ=1.5418 Å)
Generator: 40 kV and 15 mA
Sample: zero background sample holders

### Method

continuous scan mode from 4° to 40°
step size=0.0217°
counting time 500 s

### Example 7: UPLC Method—Assay Method

The release profile of the sample was analysed using an AT Xtend semi-automatic dissolution bath (Sotax) coupled CP piston pump (Sotax). An USP II (paddle) method was used with FaSSIF (pH 6.5) as dissolution medium at a temperature of 37° C. The paddle was rotated at 50 rpm. Approximately 10 mg of UA was brought into 1000 ml of medium representing a maximum concentration of 0.01 mg/ml. Samples (i.e. 1.5 mL) of the medium were taken at set time points by a piston pump directly into UPLC vials. No dilution step was required, these samples were injected as such. Each sample was analysed in triplicate. Concentration was calculated by a calibration curve of UA, which was made in organic medium (dissolving in DMSO and dilution with ACN).

Chromatographic conditions:

| System: | UPLC | |
|---|---|---|
| Detector: | PDA (250-350 nm) - optimal | |
| | wavelength: 230 nm | |
| Column: | SunFire C18 column, 100 A, | |
| | 3.5 μm, 4.6 × 150 mm | |
| Flow rate: | 1.2 | mL/min |
| Column temperature: | 40° | C. |
| Injection volume: | 7 | μl |
| Run time: | 25 | min |

| | |
|---|---|
| Needle wash (Sample | MeOH/H2O (9/1, v/v) |
| manager wash): | |
| Seal wash: | MeOH/H2O (3/7, v/v) |
| Purge solvent (Sample | MeOH/H2O (3/7, v/v) |
| manager purge): | |
| Mobile phase: | A: 0.05% TFA (w/v) in H2O |
| | B: 0.05% TFA (w/v) in ACN |

| Time | 0 | 2.0 | 10 | 12 | 12.1 | 15 |
|---|---|---|---|---|---|---|
| % A | 95 | 95 | 10 | 10 | 95 | 95 |
| % B | 5 | 5 | 90 | 90 | 5 | 5 |

### Example 8: Dissolution Via UPLC—10 mg UA Dose

The release profile of the sample was analysed using an AT Xtend semi-automatic dissolution bath (Sotax) coupled CP piston pump (Sotax). An USP II (paddle) method was used with FaSSIF (pH 6.5) or demineralised water as dissolution medium at a temperature of 37° C. The paddle was rotated at 50 rpm. Approximately 10 mg of UA was brought into 1000 ml of medium representing a maximum concentration of 0.01 mg/ml. Samples (i.e. 1.5 mL) of the medium were taken at set time points by a piston pump directly into UPLC vials. No dilution step was required, these samples were injected as such. Each sample was analysed in triplicate. Concentration was calculated by a calibration curve of UA, which was made in organic medium (dissolving in DMSO and dilution with ACN).

Chromatographic conditions:

| System: | UPLC | |
|---|---|---|
| Detector: | PDA (250-350 nm) - optimal | |
| | wavelength: 230 nm | |
| Column: | SunFire C18 column, 100 A, | |
| | 3.5 μm, 4.6 × 150 mm | |
| Flow rate: | 1.2 | mL/min |
| Column temperature: | 40° | C. |
| Injection volume: | 7 | μl |
| Run time: | 25 | min |

| | |
|---|---|
| Needle wash (Sample | MeOH/H₂O (9/1, v/v) |
| manager wash): | |
| Seal wash: | MeOH/H₂O (3/7, v/v) |
| Purge solvent (Sample | MeOH/H₂O (3/7, v/v) |
| manager purge): | |
| Mobile phase: | A: 0.05% TFA (w/v) in H2O |
| | B: 0.05% TFA (w/v) in ACN |

| Time | 0 | 2.0 | 10 | 12 | 12.1 | 15 |
|---|---|---|---|---|---|---|
| % A | 95 | 95 | 10 | 10 | 95 | 95 |
| % B | 5 | 5 | 90 | 90 | 5 | 5 |

### Example 9: Urolithin a (UA) Spray Dried Alone

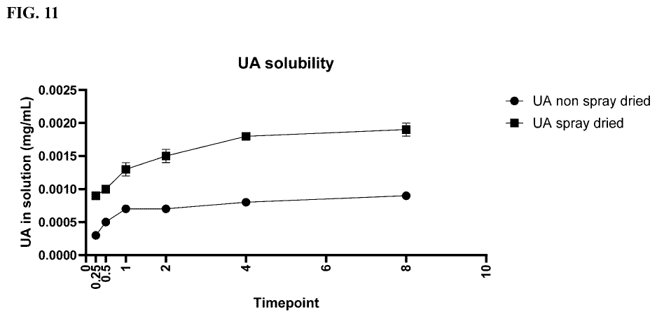

We tested whether spray drying UA using water as solvent as a strategy to improve UA solubility. UA was dissolved in water to form a stable suspension. Suspension was stirred minimal for 1 hour. Following spray drying, the UA spray dried formulation was analyzed for their solubility when dissolved in a biorelevant medium recapitulating a gut-like environment (FaSSIF, ph=6.5). Solubility of the UA spray dried formulations was compared to solubility of pure—non spray dried—UA powder. For both pure formulations, an equivalent dose of 10 mg UA was added to FaSSIF medium. Solubility was analysed by UPLC method (Ultra-Performance Liquid Chromatography) at different time points after addition of powders to the FaSSIF medium. UA spray dried powder showed a strikingly enhanced solubility profile, when compared to pure UA powder (FIG. 11).

### Example 10: Urolithin a (UA) Spray Dried with Arabic Gum

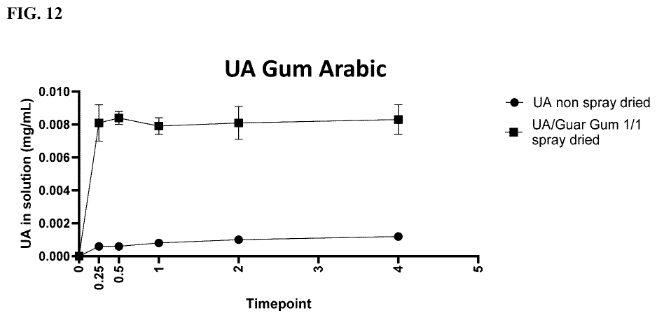

We further tested UA based formulation using water as solvent and spray dried with Arabic gum. UA was dissolved in water to form a stable suspension. Arabic gum (lot number 3413117739, Roth) was dissolved in water to form a solution. The UA suspension in water was complexed with Arabic gum at 1/1 ratio. Following spray drying, the resulting UA/Arabic gum powder formulation analyzed for their solubility when water. Solubility of the UA/Arabic gum formulations were compared to solubility of pure UA powder. For both pure UA powder and UA/Arabic gum formulations, an equivalent dose of 10 mg UA was added to water. Solubility was analysed by UPLC method (Ultra-Performance Liquid Chromatography) at different time points after addition of powders to the water. UA/Arabic gum powders showed a strikingly enhanced solubility profile, when compared to pure UA non spray dried powder (FIG. 12).

### Example 11: Urolithin a (UA) Spray Dried with Arabic Gum Solubility in Water

We evaluated the effect of spray drying UA with food-grade Arabic gum (Willy Benecke type 4810) on UA solubility in water. UA was dissolved in water to form a stable suspension. Arabic gum was dissolved in water to form a solution. The UA suspension was combined with Arabic gum solution at different weight ratios (UA/Arabic gum: 2/1, 4/1, and 9/1). Each mixture was spray dried using the same protocol as Example 12 below. For each formulation, an equivalent dose of 10 mg UA was added to 1000 mL of demineralized water (pH≈6.5). Dissolution was monitored over 72 hours using UPLC (Ultra-Performance Liquid Chromatography) at 230 nm. The release profile of UA was compared to non-spray-dried UA powder.

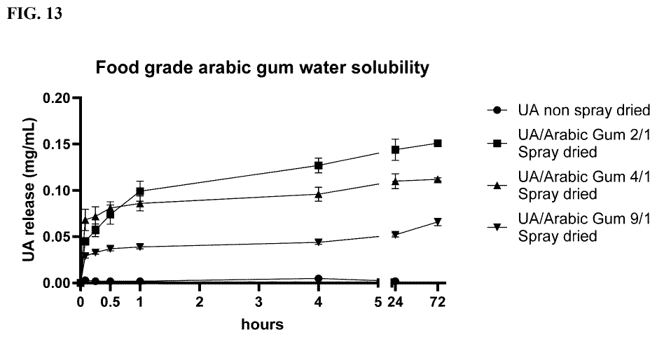

Spray drying UA with Arabic gum significantly improved UA solubility compared to non-spray-dried UA (FIG. 13). For instance, at 4 hours, UA release reached approximately:
UA/Arabic gum 2/1: ~0.15 mg/mL
UA/Arabic gum 4/1: ~0.10 mg/mL
UA/Arabic gum 9/1: ~0.06 mg/mL versus non-spray-dried UA: ~0.01 mg/mL

The improvement was evident across all ratios tested, with the 2/1 ratio showing the highest solubility enhancement. Spray drying with Arabic gum enabled UA to achieve up to 15-fold higher solubility compared to non-spray-dried UA (FIG. 13).

To assess whether the solubility improvement was due to spray drying rather than simply combining UA with Arabic gum, we prepared physical mixtures of UA and Arabic gum at ratios 2/1 and 4/1 (w/w) without spray drying. Physical mixtures were prepared by blending UA and Arabic gum powders at the same ratios using mortar and pestle until visually homogeneous. These mixtures were tested under identical conditions as the spray-dried samples (10 mg UA in 1000 mL demineralized water, monitored over 72 hours using UPLC).

At 24 hours, spray-dried UA/Arabic gum 2/1 reached ~0.15 mg/mL UA release, whereas the physical mixture remained near baseline (~0.01 mg/mL).

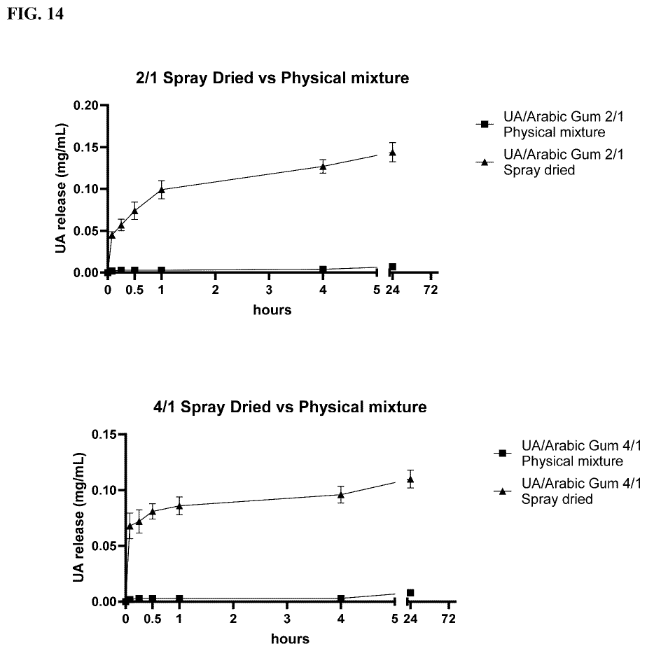

Similarly, spray-dried UA/Arabic gum 4/1 achieved ~0.10 mg/mL UA release, while the physical mixture showed negligible improvement (FIG. 14).

This comparison demonstrates that the solubility enhancement is not attributable to Arabic gum alone but to the spray-drying process, which transforms UA from its crystalline state into an amorphous dispersion within the gum matrix. The inventive concept lies in:
Recognizing that Arabic gum, when combined with spray drying, creates a unique structural environment that dramatically improves UA water solubility.
Showing that simple physical mixing fails to achieve any meaningful solubility increase, confirming the critical role of spray drying in achieving the technical effect.

This finding is unexpected because Arabic gum is traditionally used as a stabilizer or emulsifier, not as a solubility-enhancing excipient for hydrophobic actives like UA. The synergy between Arabic gum and spray drying represents a novel and non-obvious approach to improving UA bioavailability.

### Example 12: Urolithin a (UA) Spray Dried with Arabic Gum Flowability and Density

To evaluate the effect of spray drying with Arabic gum on powder handling characteristics, i.e. flowability and density, we compared non-spray-dried UA powder to a spray-dried UA/Arabic gum formulation (ratio 4/1 w/w). The invention provides a spray-dried UA/Arabic gum formulation exhibiting improved powder handling characteristics, including enhanced flowability (from 12.8 mg/s to 47.7 mg/s) and increased bulk density (from 0.39 g/mL to 0.50 g/mL) compared to non-spray-dried UA. Specifically, spray drying improved flowability by nearly 4-fold and increased bulk density by ~28%.

| | | Spray dried 4/1 |
|---|---|---|
| Analyses | Non-spray dried UA | UA/Arabic gum |
| Flowability (mg/s) | 12.8 | 47.7 |
| Bulk density | 0.39 | 0.50 |

This improvement is unexpected because Arabic gum is not typically employed to modify powder flow or density in formulations containing hydrophobic actives. The inventive step lies in combining Arabic gum with spray drying to achieve dual benefits—bioavailability enhancement and superior manufacturability—addressing critical challenges in nutraceutical and pharmaceutical production.

### Methods

Flowability was assessed using a FlowPro instrument. A sample containing unit (SCU) with a 3 mm opening was filled with the test powder and placed in the hopper. The hopper was set to oscillate at a frequency of one up-and-down movement per second, allowing the powder to discharge through the SCU opening. The powder fell onto a balance connected to the system, which continuously recorded the mass flow rate in milligrams per second (mg/s). A small amount of product was weighted into a 10 mL—graduated cylinder and the volume was checked to determine the bulk density.

### Example 13: Urolithin a (UA) Spray Dried with Arabic Gum Stability

We then investigated the stability of the UA/Arabic gum powder produced as in the examples above. Stability was assessed by comparing, water solubility and flowability of powder at time 0 (T0) with water solubility and flowability after 3 months (T3M) at:
25 C, 60% RH, i.e. long-term stability condition defined by ICH guidelines (RH=relative humidity) or
40 C, 75% RH—i.e. accelerated stability testing condition.

Results show that powder properties remained unchanged or even improved, indicating that the spray dried formulation is stable at both long-term stability condition and accelerated stability testing condition.

| | | Solubility (mg/ml | Flowability |
|---|---|---|---|
| Timepoint | Condition | after 24 h) | (mg/s) |
| T0 | N/A | 0.076 | 47.7 |
| T3M | 25 C., 60% RH | 0.083 | 53.6 |
| T3M | 40 C., 75% RH | 0.076 | 54.6 |

### Example 14: Spray Drying UA with Pectin Synergistically Enhances Mitochondrial Respiration in Muscle Cells

In this example we investigated the biological effects of a formulation spray drying Urolithin A (UA) with pectin on mitochondrial function compared to UA and pectin administered separately. The UA/Pectin formulation was prepared by spray drying UA with pectin at a weight ratio of 4:1 (UA/pectin) using the protocol described in Example 4. Cells were treated with
DMSO as control,
the spray dried powder consisting of UA/pectin at a ratio 4:1, to expose cells to an UA equivalent dose of 50 uM
Urolithin A alone at a dose of 50 uM
Pectin alone at a dose equivalent to the dose in the UA/Pectin spray dried experimental condition (i.e ¼ w/w)

Treatments were administered for 24 h. Mitochondrial respiration was assessed by measuring oxygen consumption rates under maximal conditions.

Treatment with pectin alone led to a modest non-significant increase in oxygen consumption by 10.93%. Treatment with UA alone led to increase in oxygen consumption by 28.01%.

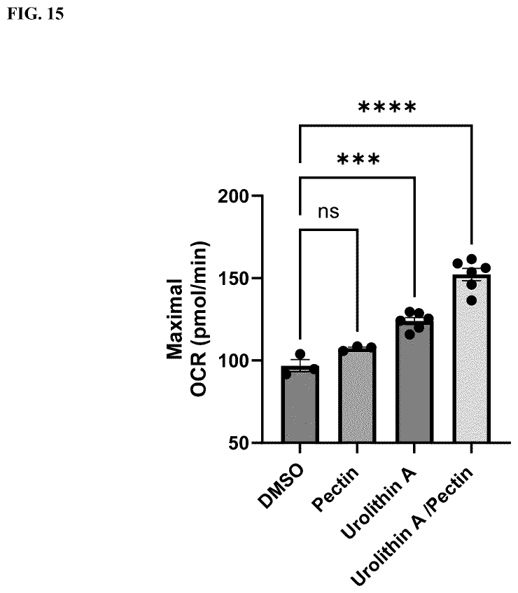

Remarkably, combining UA/Pectin together resulted in a statistically significant and synergistic enhancement of 57.11% (FIG. 15, Table 1).

### Table 1.

Table shows the percentage change in maximal respiration from data in FIG. 15. Statistical test used is One-Way ANOVA. Ns=non significant.

| | % increase max | Adjusted |
|---|---|---|
| | mitochondrial | P |
| Comparison | respiration | Value |
| Pectin vs DMSO | 10.93% | ns |
| Urolithin A vs DMSO | 28.01% | *** |
| Urolithin A/Pectin vs DMSO | 57.11% | **** |

These findings reveal an unexpected and novel synergy between UA and pectin when combined via spray drying. Spray drying creates an amorphous dispersion that enhances UA bioavailability and functional activity, which is not achieved by simple co-administration. The combined effect on mitochondrial respiration indicates a promising strategy for enhancing muscle cellular health. This synergy has clinically relevant implications for interventions targeting conditions associated with mitochondrial dysfunction.

### Example 15: Pectin Formulations with Glycerol

We developed the following gummy formulations containing increasing amounts of glycerol in order to identify the optimal amount of glycerol for gummy properties.

| Material Description | Formula 1 | Formula 2 | Formula 3 | Formula 4 | Formula 5 |
|---|---|---|---|---|---|
| Frutalose L90 | 36.06 | 36.06 | 36.06 | 36.06 | 36.06 |
| FiberSMART ® OTS90 SOLUBLE | 35.64 | 33.64 | 31.64 | 29.64 | 25.64 |
| ORGANIC TAPIOCA FIBER | | | | | |
| D. I. WATER | 9.53 | 9.53 | 9.53 | 9.53 | 9.53 |
| UROLITHIN A | 9.35 | 9.35 | 9.35 | 9.35 | 9.35 |
| NAT STRAWBERRY WONF | 3.01 | 3.01 | 3.01 | 3.01 | 3.01 |
| JAMMY TYPE | | | | | |
| GENU ® PEKTIN TYPE LM-104 AS | 2.5 | 2.5 | 2.5 | 2.5 | 2.5 |
| VEGETABLE JUICE LIQUID | 1.36 | 1.36 | 1.36 | 1.36 | 1.36 |
| SODIUM CITRATE DIHYDRATE | 0.818 | 0.818 | 0.818 | 0.818 | 0.818 |
| EXBERRY ® Shade “Purple Plum” | 0.67 | 0.67 | 0.67 | 0.67 | 0.67 |
| D. I. WATER | 0.662 | 0.662 | 0.662 | 0.662 | 0.662 |
| MALIC ACID FINE GRANULAR | 0.33 | 0.33 | 0.33 | 0.33 | 0.33 |
| SODIUM CITRATE DIHYDRATE | 0.07 | 0.07 | 0.07 | 0.07 | 0.07 |
| Stevia Reb M 95%- | 0 | 0 | 0 | 0 | 0 |
| Bioconversion | | | | | |
| Veg glycerine | 0 | 2 | 4 | 6 | 10 |
| Total | 100 | 100 | 100 | 100 | 100 |

In order to evaluate the characteristics of the gummy compositions mechanical properties of the gummies were measured using a texture-analysis instrument configured to determine compression, penetration and Overall texture results.

Increasing glycerol concentration in the formulation produced a dose-dependent increase in all measured texture parameters. Compression firmness (Cfirmness), defined as the force required to compress the gummy, and compression toughness (Ctoughness), defined as the work required for compression, both increased with glycerol content. Similarly, penetration firmness (Pfirmness), the force needed to puncture the gummy, and penetration toughness (Ptoughness), the work required to continue puncture, showed proportional increases.

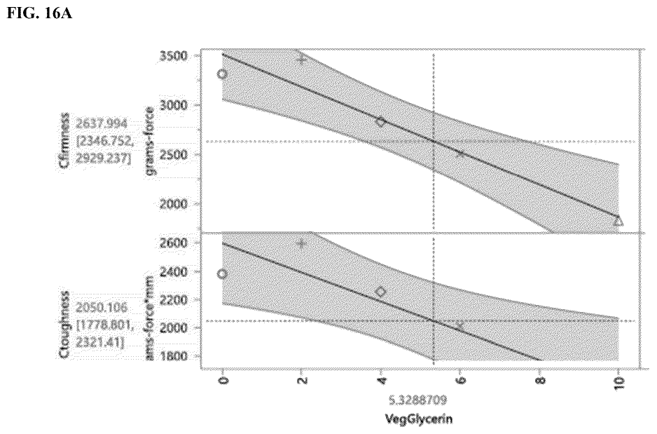

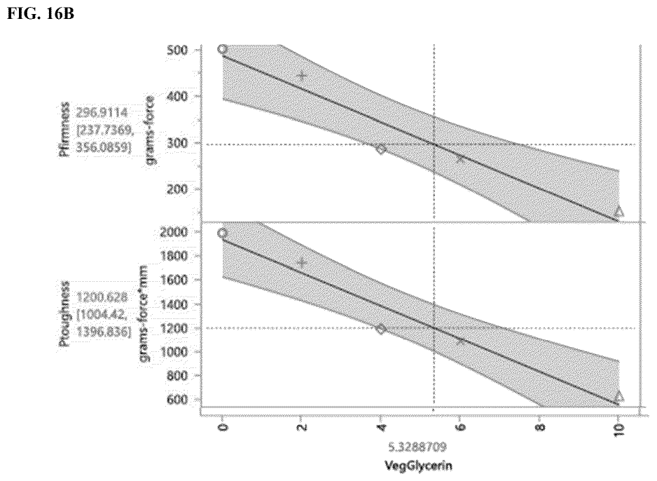

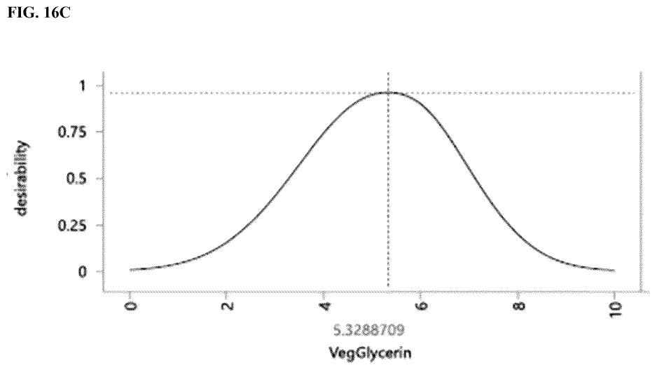

Stickiness (Pstickiness), measured as the maximum adhesive force of the gummy material on the probe, also rose with glycerol concentration. These results demonstrate that glycerol enhances firmness, toughness, and stickiness in a concentration-dependent manner (FIGS. 16A and 16B).

We correlated the amount of glycerin with gummy desirability to consumer and identified the optimal dose of glycerin at 5.3%. (FIG. 16C)

### Methods

### The Compression Test:

Cfirmness and Ctoughness are measures of the force (grams-force) and work (grams-force*mm) required, respectively, for compression of the gummies with a 12 mm diameter flat probe.

### Penetration Test

Pfirmness, Ptoughness, and Pstickiness, are measures of, while using a 2 mm probe, the force (grams-force) required to initially puncture the gummy, the work (grams-force*mm) required to continue to puncture the gummy, and the maximum adhesive force of the internal gummy material on the probe (grams-force).

### EQUIVALENTS

The invention has been described broadly and generically herein. Those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic document.

1. A formulation comprising:
(a) a compound of formula (I) or a salt thereof: 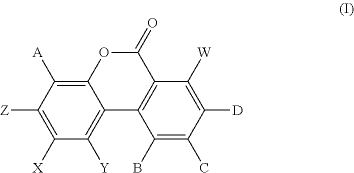
wherein:
A, B, C and D are each independently selected from H and OH;
W, X and Y are each independently selected from H and OH; and
Z is selected from H and OH; and
(b) Gum Arabic,
wherein the ratio of the compound of formula (I) to Gum Arabic is between about 1 to 4 (w/w) and about 15 to 1 (w/w).

2. The formulation as claimed in claim 1, wherein ratio of the compound of formula (I) to Gum Arabic is between:
(i) about 1 to 5 and about 10 to 1 (w/w);
(ii) about 1 to 4 to about 10 to 1 (w/w);
(iii) about 1 to 2 to about 10 to 1 (w/w)
(iv) about 1 to 1 to about 10:1 (w/w);
(v) about 2 to 1 to about 8:1 (w/w);
(vi) about 2 to 1 to about 6:1 (w/w); or
(vii) about 3 to 1 to about 5 to 1 (w/w).

3. The formulation as claimed in claim 1, wherein the Gum Arabic is selected from the exudate, for example, dried exudate, from the trunks and/or branches of Acacia Senegal, Vachellia (Acacia) seyal, Acacia polyacantha, Acacia laeta and/or Acacia mellifera, for example, the exudate is from the trunks and/or branches of Acacia Senegal and/or Vachellia (Acacia) seyal.

4. The formulation as claimed in claim 1, comprising:
a. About 9% to about 94% (w/w) of a compound of formula (I) or a salt thereof; and
b. about 6% to about 91% Gum Arabic.

5. A formulation comprising:
a. a compound of formula (I) or a salt thereof: 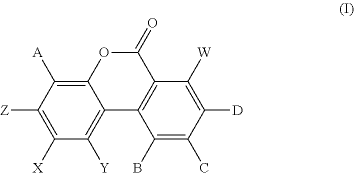
wherein:
A, B, C and D are each independently selected from H and OH;
W, X and Y are each independently selected from H and OH; and
Z is selected from H and OH;
b) pectin; and
c) glycerol.

6. The formulation as claimed in claim 4, wherein the pectin is selected from low methoxy pectin, high methoxy pectin or amidated low methoxy-pectin, or a mixture thereof, for example, wherein the pectin is low methoxypectin, such as, amidated low methoxypectin.

7. The formulation as claimed in claim 4, wherein the formulation comprises a ratio of the compound of formula (I) to pectin to glycerol in the range between about 3 to about 1 to 2 (w/w) to about 5 to about 1 to about 2 (w/w).

8. The formulation of claim 5, comprising:
a. about 1% to about 20% (w/w) a compound of formula (I) or a salt thereof;
b. about 0.1% to about 10% (w/w) pectin; and
c. about 2% to about 8% (w/w) glycerol, for example about 5%.

9. (canceled)

10. (canceled)

11. The formulation, as claimed in claim 1, further comprising:
c). glycerol.

12. The formulation as claimed in claim 8, wherein the formulation comprises glycerol in the range about 4% to about 6% (w/w), for example about 5% (w/w).

13. The formulation as claimed in claim 8, wherein the formulation comprises a compound of Formula (I) in the range about 5% to about 15%.

14. The formulation as claimed in claim 5, further comprising:
d). soluble fibre, for example, about 20% (w/w) to about 75% (w/w) soluble fibre.

15. The formulation as claimed in claim 14, further comprising:
e). one or more acids, for example, one or more acids selected from citric acid or malic acid for example, one or more acids in the range about 0.1% to about 1% (w/w).

16. The formulation as claimed in claim 15, further comprising:
f). a buffer.

17. The formulation, as claimed in claim 5, comprising:
a. About 1% to about 20% (w/w) of a compound of formula (I) or a salt thereof, as defined in claim 1; for example, urolithin A;
b. about 0.1% to about 10% (w/w) pectin;
c. about 2 to about 8% glycerol; and
d. about 20% (w/w) to about 75% (w/w) soluble fibre; or
a. about 0.5% (w/w) to about 5% (w/w) pectin, for example, about 1.25% (w/w) to 3% (w/w) pectin, for example, amidated low-methoxy pectin:
b. about 1% (w/w) to about 20% (w/w) of a compound of formula (I) or a salt thereof, as defined in claim 1; for example, urolithin A;
c. about 2% (w/w) to about 8% (w/w) glycerol, for example about 4% (w/w) to about 6% (w/w) glycerol; and
d. about 20% (w/w) to about 75% (w/w) soluble fibre.

18. (canceled)

19. The formulation, as claimed in claim 4, comprising:
a. About 1% to about 20% (w/w) of a compound of formula (I) or a salt thereof, as defined in claim 1; for example, urolithin A;
b. about 0.1% to about 10% (w/w), Gum Arabic;
c. about 2 to about 8% glycerol; and
d. about 20% (w/w) to about 75% (w/w) soluble fibre.

20. The formulation as claimed in claim 1, wherein the compound of formula (I) is selected from one or more of the group selected from urolithin A, urolithin B, urolithin C and urolithin D.

21.-38. (canceled)

39. A method of:
a. increasing or maintaining mitochondrial function;
b. treating, preventing, or managing a mitochondria-related condition associated with altered mitochondrial function or reduced mitochondrial density; or
c. treating, preventing, or managing a mitochondria-related disease;
comprising administering to a subject in need thereof an effective amount of the composition of claim 1.

40. A non-therapeutic method of:
a. enhancing muscle performance;
b. improving, maintaining or reducing the loss of muscle function;
c. increasing muscle strength, increasing or maintaining muscle mass or muscle recovery;
d. improving sports performance, physical performance and/or endurance capacity;
e. inhibiting or retarding physical fatigue, enhancing working capacity and endurance and reducing muscle fatigue; or
f. enhancing performance, physical recovery; and/or physical endurance;
comprising administering to a subject in need thereof an effective amount of the composition of claim 1.

41. A non-therapeutic method of:
a. enhancing physical performance;
b. enhancing physical recovery;
b. enhancing physical endurance;
d. maintaining or enhancing skin health and/or reducing the impact of chronological aging or biological aging;
e. maintaining or enhancing hair health;
f. maintaining or enhancing immune health;
g. maintaining or enhancing brain health;
h. maintaining or enhancing recovery after treatment;
i. increasing healthspan; or
j. slowing aging and/or increasing longevity,
comprising administering to a subject in need thereof an effective amount of the composition of claim 1;
wherein enhancing physical performance comprises at least one effect selected from the group consisting of enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage;
wherein enhancing physical recovery comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage; and
wherein enhancing physical endurance comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage.
